(19) United States
(12) Patent Application Publication
(10) Pub. No.: US 2020/0283728 A1
(43) Pub. Date: Sep. 10, 2020

(54) MODIFIED T CELLS AND USES THEREOF

(71) Applicant: MUSC FOUNDATION FOR RESEARCH DEVELOPMENT, Charleston, SC (US)

(72) Inventors: Chrystal M. PAULOS, Charleston, SC (US); Kinga MAJCHRZAK, Charleston, SC (US); Jacob S. BOWERS, Charleston, SC (US)

(73) Assignee: MUSC FOUNDATION FOR RESEARCH DEVELOPMENT, Charleston, SC (US)

(21) Appl. No.: 16/479,740

(22) PCT Filed: Jan. 25, 2018
(86) PCT No.: PCT/US2018/015314

§ 371 Date: Jul. 22, 2019

Related U.S. Application Data
(60) Provisional application No. 62/450,255, filed on Jan. 25, 2017.

(51) Int. Cl. C12N 5/0783; A61K 35/17; A61P 35/00

(52) U.S. Cl. CPC C12N 5/0636; A61K 35/17; A61P 35/00; C12N 2501/415; C12N 2501/727; C12N 2501/2301; C12N 2501/15; C12N 2501/2321; C12N 2501/2304; C12N 2501/2323; C12N 2501/24; C12N 2501/2302; C12N 2501/2306

(57) ABSTRACT

Provided herein are methods for the production of enhanced ICOS-stimulated Th17 cells by co-stimulation with inducible coactivator (ICOS) and an inhibitor of POK/Akt signaling and/or an inhibitor of Wnt/β-catenin signaling. Further provided are methods for treatment of cancer by administration of the enhanced ICOS-stimulated Th17 cells as an adoptive T cell therapy.

This application claims the benefit of U.S. Provisional Patent Application No. 62/450,255, filed Jan. 25, 2017, the entirety of which is incorporated herein by reference.

The invention was made with government support under Grant Nos. R01 CA175061, R01 CA208514, F30 CA200272, T32 GM008716 and UL1 TR000062 awarded by the National Institutes of Health. The government has certain rights in the invention.

### BACKGROUND OF THE INVENTION

### 1. Field of the Invention

The present invention relates generally to the field of immunology and medicine. Particularly, it concerns methods and compositions for treating cancer, such as by the administration of modified Th17 cells.

### 2. Description of Related Art

Th17 cells are a unique CD4⁺ T cell subpopulation, which mediate robust tumor immunity upon transfer into preclinical tumor models (1-3). Th17 cells are characterized by their capacity to secrete inflammatory cytokines IL-17A, IL-17F and IL-22 and surface expression of IL-23 receptor (IL-23R) (4). Th17 cell development and function is controlled by transcription factors such as RORγt, RORα, cMaf and STAT3 (5), whereas IL-23 signaling is crucial for their maintenance (6). Although Th17 cells exhibit a terminally differentiated phenotype, indicated by low CD62L expression, they demonstrated “stemness” qualities manifested by self-renewal, multi-potency and persistence (3). Th17 cells accumulate β-catenin and show high Tcf7 and Lef1 expression, downstream target genes of the Wnt/β-catenin pathway, previously associated with the self-renewal potential of hematopoietic stem cells (HSCs) (7). Th17 multi-potency is suggested since they can give rise to Th1-like cells, which express T-bet and produce IL-17A and IFN-γ simultaneously (3). Moreover, Th17-derived cells engraft better and are resistant to apoptosis (8). Unlike Th1 cells, Th17 cells preserved a stem cell-like molecular signature, which allow them to serve as an inexhaustible source of effector cells able to eradicate tumors. Consequently, Th17 cells more effectively regress melanoma in preclinical model than Th1 cells (3,8).

The phosphoinositide 3-kinase (PI3K) pathway is primarily involved in cell proliferation in response to multiple stimuli (hormones, growth factors etc.). In T lymphocytes, this pathway is activated by TCR signaling, IL-2 and co-stimulation (particularly ICOS) (10,11). Activated PI3K converts phosphatidylinositol(4,5)-diphosphate (PIP2) to phosphatidylinositol(3,4,5)-triphosphate (PIPS), followed by phosphorylation of serine/threonine kinase Akt. Upon activation, Akt regulates a wide range of downstream effectors leading to metabolic changes and proliferation. The members of class IA PI3Ks family are heterodimers composed of a catalytic subunit p110α, p110β or p110δ and a regulatory subunit p85α, p55α, p50α, p85β or p55γ (12).

Key roles for PI3K3 in T cells were previously shown using kinase-inactivated p110δ^{D910A} mice, p110δ knockout mice or the small molecule inhibitor IC87114 (13,14). Suppression of PI3K3 impairs the differentiation of Th1, Th2, Th17 or Tfh subsets and significantly decreases their production of cytokines (IFN-γ, IL-4, IL-17 and IL-21, respectively) (15,16). PI3Kδ is also important for the expression of adhesion molecules and chemokine receptors in antigen-dependent trafficking of T cells and most recently for interaction of T cells with antigen presenting cells (13,17). ICOS is a potent activator of PI3K pathway, since ICOS has a unique YMFM SH2 binding motif that recruits a PI3Ks. ICOS preferentially recruits the p50α regulatory subunit, which has superior kinase activity than classically recruited p85 regulatory subunit, thus ICOS strongly induces Akt signaling (11,18,19).

The Wnt/β-catenin pathway is crucial not only for thymocyte differentiation but also for T cells development by tuning cell survival, migration and lineage fate decisions (20). In HSCs, this pathway is responsible for self-renewal and sustainment in an undifferentiated state. However constitutive activation of β-catenin alone paradoxically induced the apoptosis of HSCs (7). Only upon simultaneous activation of the PI3K/Akt and Wnt/β-catenin pathways, HSCs demonstrated long-term expansion and self-renewal caused by enhanced proliferation and decreased apoptosis (PI3K/Akt activation) as well as blocked differentiation (β-catenin activation) (7). However, there is an unmet need to determine the mechanism by which ICOS-activated Th17 cells maintained their effectiveness for the development of enhanced T cell therapies.

### SUMMARY OF THE INVENTION

In a first embodiment there is provided a method (e.g., an in vitro method) for producing enhanced ICOS-stimulated Th17 cells comprising: (a) obtaining a starting population of ICOS-stimulated Th17 cells; and (b) culturing the Th17 cells in the presence of an inhibitor of PI3K/Akt signaling and/or an inhibitor of Wnt/β-catenin signaling, thereby obtaining enhanced ICOS-stimulated Th17 cells. In some aspects, culturing of step (b) is for 4 to 10 days, such as for 5, 6, 7, 8, 9, 10 or more days.

In a second embodiment, there is provided an in vitro method for producing enhanced immune cells comprising: (a) obtaining a starting population of immune cells; and (b) culturing the cells in the presence of an inhibitor of PI3K/Akt signaling and/or an inhibitor of Wnt/I3-catenin signaling, thereby obtaining enhanced immune cells. In some aspects the immune cells comprise T cells, NK cells or macrophages. In some aspects, the immune calls comprise CD4⁺ T cells or CD8 T cells. In some aspects, the immune cells comprise Th17 cells. In some aspects, the immune cells are cultured in the presence of an inhibitor of PI3K/Akt signaling and an inhibitor of Wnt/β-catenin signaling.

In another embodiment, the present disclosure provides an engineered immune cell comprising a genetic disruption of a gene in the PI3K/Akt pathway and/or the Wnt/β-catenin pathway. In some aspects, the engineered immune cell comprises a genetic disruption of a gene in the PI3K/Akt pathway and the Wnt/β-catenin pathway. In some aspects, the engineered cell is a T cell, NK cell, or macrophage. In some aspects, the engineered cell is a CD4⁺ T cell or CD8⁺ T cell. In some aspects, the genetic disruption was produced with a zinc finger nuclease, a transposase or a CRISPR construct.

In yet another embodiment, the disclosure provides an in vitro method for producing enhanced inducible costimulator (ICOS)-stimulated Th17 cells comprising: (a) obtaining a starting population of ICOS-stimulated Th17 cells; and (b) culturing the Th17 cells in the presence of an inhibitor of PI3K/Akt signaling and/or an inhibitor of Wnt/β-catenin signaling, thereby obtaining enhanced ICOS-stimulated Th17 cells.

In some aspects, the inhibitor of PI3K/Akt signaling is an inhibitor of p110δ. In further aspects, the inhibitor can be an agent that reduces expression of a gene in the PI3K/Akt pathway. For example, the inhibitor can be a siRNA, short hairpin RNA or an antisense nucleic acid. In further aspects, the inhibitor is an agent that disrupts a gene in the PI3K/Akt pathway, such as a zinc finger nuclease, a transposase or a CRISPR construct. In particular aspects, the inhibitor of p110δ is CAL-101 (also known as Idelalisib and GS-1101). In certain aspects, the CAL-101 is present at a concentration of 5 to 15 μM, such as 6, 7, 8, 9, 10, 11, 12 13, 14, 15 μM, or higher. In some aspects, the p110δ inhibitor is CAL-101, PIK-294, PI-3065, PIK-293, IC-87114, Duvelisib (IPI-145, INK1197), Omipalisib (GSK2126458, GSK458), PF-04691502, GSK1059615, VS-5584 (SB2343), Pictilisib (GDC-0941), PI-103, ZSTK474, Apitolisib (GDC-0980, RG7422), BEZ235 (NVP-BEZ235, Dactolisib), SAR245409 (XL765), BKM120 (NVP-BKM120, Buparlisib), LY294002, or any combination thereof.

In certain aspects, the inhibitor of Wnt/β-catenin signaling is an inhibitor of (3-catenin. In further aspects, the inhibitor can be an agent that reduces expression of a gene in the Wnt/β-catenin pathway. For example, the inhibitor can be a siRNA, short hairpin RNA or an antisense nucleic acid. In further aspects, the inhibitor is an agent that disrupts a gene in the Wnt/β-catenin pathway, such as a zinc finger nuclease, a transposase or a CRISPR construct. In specific aspects, the inhibitor of β-catenin is indomethacin. In certain aspects, the indomethacin is present at a concentration of 50 to 100 μM, such as 55, 60, 65, 70, 75, 80, 85, 90, 95, 100 μM or higher. In some aspects, the β-catenin inhibitor is Indomethacin, FH535, PNU-74654, iCRT 14, TAK 715, JW 74, JW 67, XAV 939, or any combination thereof. In certain aspects, the β-catenin inhibitor is a nonsteroidal anti-inflammatory drug (NSAID) such as aspirin.

In some aspects, the enhanced ICOS-stimulated Th17 cells exhibit an increased ability for engraftment, persistence, and/or antitumor activity in vivo. In certain aspects, the enhanced ICOS-stimulated Th17 cells have decreased expression of RORγt, cMaf and/or STAT-3. In some aspects, the enhanced ICOS-stimulated Th17 cells have an increased percentage of CD44^{high},CD62L^{high} cells as compared to the starting population of ICOS-stimulated Th17 cells.

In certain aspects, obtaining a starting population of ICOS-stimulated Th17 cells comprises programming T cells to a Th17 phenotype and stimulating the Th17 cells with ICOS. In some aspects, programming comprises culturing the cells in the presence of IL-1β, IL-6, IL-21, TGFβ, IL-4, IFNγ, IL-2, and/or IL-23. In particular aspects, the T cells are CD4⁺ and/or CD8⁺ T cells. In certain aspects, stimulating with ICOS comprises culturing the population of Th17 cells in a culture comprising anti-ICOS coated beads. In some aspects, the cell are further stimulated with one or more co-stimulatory agents selected from the group consisting of 41BB, CD28, CD40L, OX40, a PD-1 inhibitor, and a CTLA4 inhibitor or any other co-stimulatory of co-inhibitor molecule. Also, cytokines, such as but not limited to IL-2, IL-7, IL-12, IL-15, IL-21, IL-23, IFN-gamma, can augment the expression or generation of Th17 cells.

In certain aspects, the beads are magnetic beads. In additional aspects, the culture further comprises anti-CD3 beads. In other aspects, the culture further comprises at least one growth factor. In a specific aspect, the at least one growth factor may be IL-2. In some particular aspects, the culturing is for 5 day to 10 days or even longer.

In some aspects, the T cells are isolated from peripheral blood, cord blood, or the spleen. In certain aspects, the T cells are isolated from peripheral blood mononuclear cells.

In certain aspects, the Th17 cells are engineered to express a T cell receptor (TCR) or chimeric antigen receptor (CAR) receptor. In some specific aspects, the TCR or CAR comprises an intracellular signaling domain, a transmembrane domain, and/or an extracellular domain comprising an antigen binding region. In certain particular aspects, the antigen binding region is an F(ab′)2, Fab′, Fab, Fv, or scFv. In still further aspects, the intracellular signaling domain may be a T-lymphocyte activation domain. In other aspects, the intracellular signaling domain comprises CD3, CD28, OX40/CD134, 4-1BB/CD137, FccRIy, ICOS/CD278, ILRB/CD122, IL-2RG/CD132, DAP molecules, CD70, cytokine receptor, CD40, or a combination thereof or any other type of costimulators/cytokines. In additional aspects, the intracellular signaling domain comprises CD3 and 4-1BB/CD137. In some aspects, the transmembrane domain comprises CD28 transmembrane domain, IgG4Fc hinge, Fc regions, CD4 transmembrane domain, the CD3 transmembrane domain, cysteine mutated human CD3 domain, CD16 transmembrane domain, CD8 transmembrane domain, or erythropoietin receptor transmembrane domain.

In certain aspects, the antigen binding region binds a tumor associated antigen. In some particular aspects, the tumor associated antigen is selected from the group consisting of tEGFR, Her2, CD19, CD20, CD22, mesothelin, CEA, CD23, CD24, CD30, CD33, CD38, CD44, EGFR, EGP-2, EGP-4, EPHa2, ErbB2, FBP, MAGE-A1, MUC1, NY-ESO-1, and MART-1.

In still further aspects, the enhanced ICOS-stimulated Th17 cells have decreased expression of FoxP3 and/or CD25. In some aspects, the enhanced ICOS-stimulated Th17 cells have a central memory phenotype. In certain aspects, the enhanced ICOS-stimulated Th17 cells are capable of long-term engraftment in a mammal, such as a human.

In another embodiment, an isolated cell population is provided that may be produced according to the methods of the embodiments and aspects described herein.

In yet a further embodiment, there is provided a method of treating cancer in a subject comprising administering an effective amount of enhanced ICOS-stimulated Th17 cells to the subject. In some aspects, at least 20, 30, 40, 50, 60, 70, 75, 80, 90, 95, 96, 97, 98, or 99 percent of the T cells are enhanced ICOS-stimulated Th17 cells. In some aspects, the enhanced ICOS-stimulated Th17 cells are produced by the methods of the embodiments. In certain aspects, the cancer is melanoma. In further aspects, the method further comprises performing total body irradiation to the subject prior to administering the enhanced ICOS-stimulated Th17 cells. In some aspects, the enhanced Th17 cells exhibit increased tumor regression as compared to the starting population of Th17 cells.

In some aspects, the enhanced ICOS-stimulated Th17 cells are autologous. In additional aspects, the method further comprises lymphodepletion of the subject prior to administration of the enhanced ICOS-stimulated Th17 cells. In further aspects, lymphodepletion comprises administration of cyclophosphamide and/or fludarabine. In other aspects, the method further comprises administering at least a second therapeutic agent. For example, the at least a second therapeutic agent may comprises CD8⁺ T cells or chemotherapy, immunotherapy, surgery, radiotherapy, or biotherapy. In a particular aspect, the immunotherapy is an immune checkpoint inhibitor. In further aspects, enhanced ICOS-stimulated Th17 cells and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion.

In certain aspects, the cancer is bladder cancer, breast cancer, clear cell kidney cancer, head/neck squamous cell carcinoma, lung squamous cell carcinoma, melanoma, non-small-cell lung cancer (NSCLC), ovarian cancer, pancreatic cancer, prostate cancer, renal cell cancer, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL) or any other type of cancer. In other aspects, the cancer may be mesothelioma, pancreatic cancer, or ovarian cancer or any other type of cancer. In some aspects, said subject is a human subject.

In certain aspects, the immunotherapy comprises adoptive transfer of a T cell population. In some aspects, the immunotherapy is treatment with an immune checkpoint inhibitor, cytokines, chemotherapy or other immune modulator. In particular aspects, the immune checkpoint inhibitor is a PD-1 inhibitor or a CTLA-4 inhibitor. In some aspects, the PD-1 inhibitor is nivolumab, or pembrolizumab or other.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D: ICOS but not CD28 co-stimulation generates memory Th17 cells with superior antitumor activity. A, Th17 cells co-stimulated with ICOS regress melanoma to a greater extent than Th17 cells stimulated with CD28. C57BL6 mice bearing s.c. B16F10 tumors established for 10 days received non-myeloablative 5Gy total body irradiation (TBI). One day later, mice received an ACT treatment regimen consisting of the adoptive transfer of 1×10⁶ cultured self/tumor reactive TRP-1 CD4⁺ T cells programmed to a Th17 phenotype (with IL-β, IL-21, IL-6, TGFβ, anti-IL-4, and anti-IFN-γ) and expanded with aCD3-beads coated with a CD28 or ICOS agonist. Tumor growth measured every 2-3 days; n=8 mice/group. Data (mean±SEM) are representative of two independent experiments. B, Th17 cells primed with ICOS but not CD28 persist in vivo for 200 days. Quantification of tumor-specific (Vβ14⁺) TRP-1 Th17 cells expanded with ICOS vs. CD28 from lymph nodes, lungs and spleen 200 days post-transfer. C, ICOS-stimulated Th17 cells secrete more IFN-γ, IL-17A and IL-21 than CD28-stimulated Th17 cells when re-activated ex vivo. Donor Th17 cells were isolated from mouse spleen 200 days post-transfer and then reactivated with B16F10 tumor. The IFN-γ, IL-21 and IL-17A production was then measured using flow cytometry. D, Th17 cells stimulated with ICOS possess durable memory response to tumors even after re-challenge with B16F10 tumor. 200 day after initial adoptive transfer, surviving mice previously treated with TRP-1 Th17 cells activated with ICOS or CD28 were re-challenged with B16F10 and monitored for tumor burden compared to mice not previously treated (i.e. No treatment).

FIGS. 2A-2H: ICOS co-stimulation induces Wnt/β-catenin and PI3K/p110δ signaling pathway in Th17 cells. A, ICOS confers a distinct gene expression profile in Th17 cells. The relative expression of Th17-associated genes in TRP-1 Th17 cells stimulated with ICOS or CD28, using qPCR analysis. Analysis was performed on listed transcripts relative to β-actin. B, ICOS induces RORγt expression in Th17 cells to a greater extent than CD28 signaling. Representative histogram of RORγt expression in TPR-1 Th17 cells stimulated with ICOS (solid line) or CD28 (dashed line) agonist, on day 8 of culture. C, Th17 cells secrete more IL-17 and IFN-γ when ligated with ICOS. Representative FACS analysis of IL-17A and IFN-γ production of TRP-1 Th17 cells stimulated with ICOS vs. CD28. D-F, ICOS induces Wnt/β-catenin and PI3K/Akt pathways in Th17 cells. Western Blot analysis of (D) β-catenin, (E) phosphoAkt expression (day 8) and (F) PI3K-p110δ expression (day 3) in TRP-1 Th17 cells stimulated with ICOS vs. CD28. G-H, ICOS Th17 cells express lower β-catenin and p110δ/Akt proteins than WT Th17 cells. Western blot analysis of (G) nuclear and cytoplasmic β-catenin expression and (H) cytoplasmic PI3K-p110δ and phosphoAkt expression on WT TRP-1 Th17 cells (stimulated with an ICOS agonist) and ICOS^{-/-} Th17 cells stimulated with an ICOS or CD28 agonist, on day 8.

FIGS. 3A-3E: Pharmacological inhibition of ICOS-induced Wnt/β-catenin and PI3K/p110δ signaling pathways alter the cytokine profile and transcription factor expression in antitumor Th17 cells. A-B, inhibition of p110δ but not β-catenin suppress IL-17A and IFN-γ production by ICOS activated Th17 cells. Representative FACS plot of (A) cytokine production (IL-17, IFN-γ, IL-2, TNF-α, IL-22) in TRP-1 CD4⁺ cells polarized towards a Th17 phenotype, co-stimulated with ICOS agonist for 8 days (Control) and expanded in the presence of specific PI3K/p110δ subunit inhibitor (CAL-101, 10 μM) and/or specific β-catenin inhibitor—Indomethacin (Indo, 60 μM). (B) Bar graphs showing quantification of cytokine production (IL-17, IFN-γ, IL-2, TNF-α, IL-22). C, blockade of both pathways slightly impairs the expansion of ICOS-activated Th17 cells. Bar graph of absolute number (×10⁶) of TRP-1 Th17 cells in the absence (Control) or presence of CAL-101 and/or Indomethacin, on day 7. Values represent mean±SEM of at least three independent experiments. *, P<0.05; **, P<0.01; ***, P<0.001. D-E, transient ablation of p110δ signalling by CAL-101 decreases the expression of RORγt, cMaf and STAT3 induced in ICOS-activated Th17 cells. (D) Representative histograms showing expression of transcription factors (RORγt, cMaf, STAT-3) and (E) Western Blot analysis of nuclear RORγt and STAT-3 expression in TRP-1 Th17 cells in the absence (control) or presence of inhibitors, as indicated (day 8).

FIGS. 4A-4I: Pharmacological inhibition Wnt/β-catenin and PI3K/p110δ signaling pathways during ICOS-mediated Th17 expansion in vitro enhanced their anti-tumor efficiency, engraftment and function in vivo. A-B, treating ICOS-activated Th17 cells with CAL-101 or Indo plus CAL-101 improve their capacity to regress tumors. (A) Average tumor growth curve and (B) Survival after transfer of 0.75×10⁶ TRP-1 CD4⁺ T cells polarized towards a Th17 phenotype stimulated with ICOS agonist and/or expanded in vitro with CAL-101, specific PI3K/p110δ subunit inhibitor (CAL-101 primed) or Indomethacin, specific β-catenin inhibitor (Indo primed). C-E, pan inhibition of PI3 Kinases with Ly294002 does not replace the therapeutic effectiveness of CAL-101 priming on ICOS-stimulated Th17 cells. (C) Average tumor growth curve and (D) Survival after transfer of 0.75×10⁶ TRP-1 Th17 cells stimulated with ICOS and expanded in vitro with Ly294002 (Ly294002 primed) and/or Indomethacin. (E) Individual tumor growth curves after transfer of 0.75×10⁶ TRP-1 ICOS-activated Th17 cells expanded in vitro with inhibitors as indicated. Cells were transferred into irradiated (5Gy TBI) mice bearing established B16F10 melanomas. Tumor growth measured every 2-3 days; n=9 mice/group. Data (mean±SEM) are representative of two independent experiments. NT—no treatment; ns—without statistical significance; *, P<0.05. F-H, ICOS-activated Th17 cells cultured with both Indo and CAL-101 engraft to a greater extent than other Th17 groups in the blood and spleen. Bar graph showing (F) percentage of tumor specific (Vβ14⁺) TRP-1 Th17 cells in the blood 6 days after transfer. Bar graph showing (G) percentage and (H) number (×10⁴) of tumor specific (Vβ14⁺) TRP-1 Th17 cells in the spleen 6 days after transfer. I, donor Th17 cells are able to secrete IL-17A, IFN-γ and IL-2, 64 days post transfer in the mice if they were originally cultured in the presence of Indo or Indo plus CAL-101. (I) Bar graph showing cytokine production in vivo of Th17 cells expanded in vitro with inhibitors, as indicated. Donor Th17 cells where isolated from the spleen and re-stimulated with PMA/Ionomycin. Data represent mean±SEM. *, P<0.05; **, P<0.01; ***, P<0.001.

FIGS. 5A-5H: Th17 cells stimulated with ICOS have a central memory phenotype and express nominal FoxP3 when treated with CAL-101. A-C, inhibition of PI3Kp110δ in ICOS-activated Th17 cells supports their central memory phenotype in vitro. (A-B) Representative FACS plots showing memory phenotype by CD44 and CD62L on ICOS co-stimulated TRP-1 Th17 cells expanded with distinct inhibitors as indicated, on day 7. CD44^{high}CD62L^{high} T cells represent central memory T cells (T_{CM}) while the CD44^{high}CD62L^{low} T cells are effector memory T cells (T_{EM}). (C) Bar graphs showing expression of central memory markers (CD62L, CCR7, CD28, CD27) and effector memory marker (CCR6) on ICOS stimulated Th17 cells in the absence (control) or presence of inhibitors, as indicated. D-E, inhibition of PI3K/p110δ subunit by CAL-101 decreases FoxP3, while inhibition of pan PI3 Kinases with Ly294002 supports FoxP3 in Th17 cultures. (D-E) Representative FACS plots showing CD4⁺ FoxP3+CD25^{high} T regulatory cells (T_{reg}) in ICOS co-stimulated Th17 cells expanded in the presence of distinct inhibitors, as indicated, before and after secondary stimulation in vitro (Re-stimulated). F-H, CAL-101 supports the memory phenotype and inhibits the regulatory phenotype of ICOS activated Th17 cells. Bar graphs showing quantification of (F) CD4+CD44^{high}CD62L^{high} T_{CM} cells, (G) CD4⁺ FoxP3⁺CD25^{high} T_{reg} cells and (H) a ratio of T_{CM} cells to T_{reg} cells in Th17 cells co-stimulated with ICOS (Control) and expanded with CAL-101 and/or Indo, on day 8 (gated on CD4⁺, vβ14⁺ cells). Data represent mean±SEM of at least three independent experiments. *, P<0.05; **, P<0.01; ***, P<0.001.

FIGS. 6A-6J: Rebound of the Wnt/β-catenin pathway supports the antitumor activity of ICOS stimulated Th17 cells primed in vitro with inhibitors. A, ICOS-activated Th17 cells initially accumulate β-catenin in the nucleus when expanded in the presence of CAL-101 whereas Indo sufficiently impairs β-catenin translocation to the nucleus. (A) Western blot analysis of nuclear β-catenin and Histone-3 (loading control) expression in ICOS stimulated Th17 cells expanded in the presence or not of CAL-101 and/or Indo (day 8). B, expression of Wnt/β-catenin pathway-associated genes is up-regulated upon secondary stimulation in ICOS activated Th17 cells primed with CAL-101 and/or Indo (B) Real-time PCR analysis of fold change gene expression of Tcf7, Lef-1 and Ctnnb1 in Th17 cells expanded with inhibitors, as indicated and re-stimulated in vitro with anti-CD3 antibody (5 μg/ml for 1 h). Fold change was calculated using AACT method. C, nuclear β-catenin is restored and Tcf-7 expression is up-regulated after in vitro re-stimulation of ICOS activated Th17 cells primed in the presence of inhibitors, as indicated. (C) Western blot analysis of nuclear β-catenin, Tcf-7 and Histone-3 (loading control) expression. D-E, ICOS activated Th17 cells primed with Indo plus CAL-101 eradicate tumor more efficiently than non-primed counterparts. (D) Waterfall plot graph showing the percentage of change in tumor area and (E) Graph showing the tumor weight upon ACT Th17 cells primed with the inhibitors, as indicated (day 14). F, ICOS activated Th17 cells expanded in vitro with Indo plus CAL-101 engraft better, acquire an effector memory phenotype and recruit NK cells into the tumor upon ACT. (F) Graphs showing quantification of CD3⁺CD4⁺Vβ14⁺ tumor-specific donor cells per 0.1 g of tumor, CD4⁺Vβ14⁺CD44^{high}CD62L^{low} T_{EFM} cells and NK cells in the tumor 14 days post ACT of ICOS activated Th17 cells primed with the inhibitors, as indicated. G-H, donor Th17 stimulated with ICOS engraft better and keep their less regulatory phenotype in vivo, when primed in vitro with Indo and CAL-101. (G) Graphs showing quantification of CD3⁺CD4+Vβ14⁺ tumor-specific donor cells and CD4⁺Vβ14⁺FoxP3⁺CD25^{high} T_{reg} cells in the spleen. (H) Representative FACS plots showing T regulatory cells (T_{reg}) in the spleen upon ACT of ICOS stimulated Th17 cells expanded in the presence of inhibitors, as indicated. Data represent individual mouse. *, P<0.05. (I) Western blot of nuclear β-catenin, RORγt, STAT3 and Histone-3 (loading control) expression. (J) IL-17 rebounds and IL-2 is elevated in Th17 inhibited cells post-secondary stimulation with tumor-specific tyrosinase peptide (2 days post reactivation, ELISA of IL-17A and IL-2). Representative of three independent experiments, Student's T test. *P<0.05. ns=not significant.

FIG. 7: A schematic illustration summarizing results on pharmacological induction of durable Th17 cell memory responses to tumors. ICOS activated Th17 cells expended in vitro in the presence of CAL-101 and Indo possess a central memory phenotype (i.e. elevated CD62L expression) and less regulatory properties (decreased FoxP3 expression). Moreover, upon antigen recall in vivo they produce multiple effector cytokines (IFN-γ, IL-2 and IL-17), exhibit elevated nuclear β-catenin and Tcf7 and augmented persistence in the host, which possess self-renewal and durable memory responses to tumors in vivo.

FIGS. 8A-8C: ICOS deficient Th17 cells become activated in vitro, yet secrete less IL-17A. A-B, ICOS^{-/-} Th17 cells do not express ICOS but CD28 expression, yet become activated, as indicated by high CD69 expression. Representative flow cytometry analysis of (A) ICOS and CD28 expression on ICOS^{-/-} and wild-type (WT) CD4⁺ T cells on day 0 and (B) CD69 on WT Th17 cells and ICOS^{-/-} Th17 cells expanded with either ICOS or CD28 agonist on day 8 of culture. C, genetic ablation of ICOS diminishes Th17 function. (C) Representative flow cytometry analysis of IL-17A by IFN-γ secretion by WT Th17 cells and ICOS^{-/-} Th17 cells expanded with either ICOS or CD28 agonist on day 8 of culture.

FIG. 9: Th17 cells deficient in ICOS produce less inflammatory cytokines such as IL-17A, IL17F, CCL20, IL-22, IL-10, IL-21, IL-4 but more IFNγ. Representative ELISA analysis of cytokine production on day 3 of culture of ICOS^{-/-} and WT Th17 cells. The experiment was performed twice with similar results.

FIG. 10: Pharmacological inhibition of pan PI3 Kinases activity and Wnt/β-catenin signaling pathway alter the cytokine profile of ICOS stimulated Th17 cells. Inhibition of pan PI3 Kinases activity suppress IL-17A and IFN-γ production, but concomitant inhibition of β-catenin increase IL-2, TNF-α, IL-22 secretion by ICOS activated Th17 cells. Representative FACS plot and quantification of cytokine production in CD4⁺ cells polarized towards a Th17 phenotype, co-stimulated with ICOS agonist for 8 days (control) and expanded in the presence of total PI3K inhibitor (Ly294002, 10 μM) and specific β-catenin inhibitor—Indomethacin (Indo, 6004). Data represent mean±SEM of at least three independent experiments. *, P<0.05; ***, P<0.001.

FIG. 11: Re-stimulation of ICOS activated Th17 cells in vitro restores their cytokine production after initial inhibition of PI3K/p110δ and Wnt/β-catenin signaling pathways. ELISA analysis of cytokine production 72 hrs after secondary re-stimulation in vitro using splenocytes pulsed with trp-1 peptide (5:1 ratio), PMA/Ionomycin or B16F10 cancer cells of ICOS stimulated Th17 cells expanded with inhibitors as indicated. Data represent mean±SEM of at least three independent experiments.

FIG. 12: Th17 cells stimulated with ICOS and primed with Indo and CAL-101 acquire an effector memory phenotype but do not induce expression of exhaustion marker PD-1 on donor and host cells. Graphs showing quantification of donor CD4⁺β14⁺ tumor-specific CD44^{high}CD62L^{low} T_{EFM} cells; donor CD4⁺Vβ14₊ PD-1⁺ and host CD8₊ PD-1⁺ T cell in the spleen 14 days post ACT of ICOS activated Th17 cells primed with the inhibitors, as indicated. Data represent individual mouse.

FIGS. 13A-L: Dual-inhibited Th17 cells directly regress tumor and do not require host NK or CD8 T cells. A-C, ICOS-activated Th17 cells cultured with Indo plus CAL-101 engraft to a greater extent than other treatments in the spleen. Bar graph showing (A) percentage and (B) number (×10⁴) of TRP-1 Th17 cells in the spleen 6 days after transfer. C. Donor Th17 cells secrete IL-17A, IFN-γ and IL-2, 64 days post transfer in the mice when primed with Indo or Indo plus CAL-101. Bar graph showing cytokine production in vivo of Th17 cells expanded in vitro with inhibitors, as indicated. Donor cells where isolated from the spleen and re-stimulated with PMA/Ionomycin. Data represent mean±SEM. Student's T test, *P<0.05; **P<0.01; ***P<0.001. D. Plot showing NK cell frequency in the tumor of mice infused with various Th17 treatments. E-H. Depletion of host NK (bottom left) or CD8 T cells (bottom right) in mice does not abrogate therapy. Individual tumor growth curves of mice treated with 0.75×10⁶ transferred TRP-1 Th17 cells expanded with TRP-1 peptide, ICOS agonist and primed in vitro with CAL-101 and Indo. Cells transferred into 5Gy TBI mice bearing B16F10 melanomas. Mice were antibody depleted of host CD8 or NK cells (100 μg/mouse) twice weekly for the entire experiment starting 2 days prior to ACT. As a control, mice given Th17 therapy were administered with an IgG isotype (upper right). I-L. Depletion of donor cells in mice re-challenged with tumor impairs ACT. Surviving mice in FIG. 5J were re-challenged with B16F10 and then antibody depleted of host CD4 cells (100 μg/mouse) or with an IgG control twice weekly for the first two weeks post re-challenge. Percent survival; n=13 mice/group, Kaplan-Meier curves compared by log-rank test. *P<0.05, As a control, naïve mice were treated with an IgG isotype control (upper right panel) or treated with a CD4 depleting antibody. Indo+CAL-101+CD4 depletion is significant different that Indo+CAL-101+IgG (P<0.05).

FIGS. 14A-G: CAL-101 priming supports a de-differentiated memory phenotype including high IL-7Ra expression on pmel-1 CD8⁺ T cells. (A) Representative flow plots of CD44 by CD62L expression on pmel-1 CD8⁺ T cells primed with vehicle, AKTi or CAL-101 after 5 days of culture; representative of 3 independent cultures. (B) MFI of CD62L, CD69 and CD44 on pmel-1 CD8⁺ T cells after 5 days of culture; n=3 independent cultures. (C) Fold increase of pmel-1 CD8⁺ T cells primed with vehicle (DMSO), AKTi, or CAL-101 5 days following antigen stimulation; n=3 independent cultures. (D) MFI of CD25 and CD127 on pmel-1 CD8⁺ T cells after 5 days of culture; n=3 independent cultures. (E-G) Frequency of donor pmel-1 T cells (infused on day 0 of treatment at 8×10⁵ cells/mouse) and extracellular markers on those cells in blood of tumor bearing B6 mice preconditioned with 5 Gy total body irradiation 7 days following treatment; n=3 mice/group from one experiment. One way repeated measures ANOVA; ns=not statistically significant, *=p<0.05, **=p<0.01, ***=p<0.001, ****=p<0.0001.

FIGS. 15A-D: CAL-101 primed pmel-1 CD8⁺ T cells exert stronger antitumor response against B16F10 tumors. (A) Frequency of donor pmel-1 T cells and extracellular markers on pmel-1 (vβ13⁺) donor T cells in tumor 7 days following treatment; n=3 mice/group. One way repeated measures ANOVA; ns=not statistically significant, *=p<0.05. Tumor burden (mm²) of (B) individual mice and (C) Average tumor burden (mm²) of treatment groups which received no T cell treatment, or 8×10⁵ pmel-1 CD8⁺ T cells primed with vehicle, AKTi, or CAL-101 ex vivo; n=10-12 mice/group in one experiment. (D) Percent survival of above treatment groups. Kaplan Meier curve analyzed by log rank test; ****=p<0.0001. (See also FIG. 20).

FIGS. 16A-D: PI310 and AKT blockade induce a central memory phenotype in human CAR CD3⁺ T cells. (A) CD44 and CD62L expression on vehicle, AKTi, or CAL-101 treated T cells from normal donor PBMC; representative of 9 donors. (B) MFI of memory markers and (C) frequency of human CD3⁺ T cells expressing TIM3, CD25 and CD127; n=9 normal donors. One way repeated measures ANOVA; ns=not significant, *=p<0.05, **=p<0.01. (D) Histogram of CD127 expression on vehicle, AKTi, or CAL-101 treated T cells compared to no stain with frequency of positive cells indicated next to legend; representative of 9 donors.

FIGS. 17A-F: CAL-101 treatment improves tumor control by human CAR T cells compared to vehicle and AKTi treatment. (A) Individual tumor curves of NSG mice given M108 subcutaneously 51 days prior were treated with 4×10⁵ CD3⁺ mesoCAR-T cells primed with vehicle, AKTi, or CAL-101; n=8-12 mice/group in one experiment. (B) Tumor weight at day 71 post-transfer. (C) Percent change in size of tumors 71 days post-treatment compared to baseline tumor measurement at time of treatment. (D) Frequency of human CD45⁺ lymphocytes within the blood of treated mice 55 days post-transfer; n=2-12 mice/group from one experiment. One way repeated measures ANOVA; ns=not statistically significant, *=p<0.05. (E) Frequency of CD62L⁺ or TIM3⁺CD3⁺ TIL from lung carcinoma after 5 weeks of growth with either two weeks of CAL-101 treatment or not, and (F) change in frequencies of CD62L⁺ or TIM3⁺CD3⁺ TIL during drug treatment with CAL-101 or without (vehicle) from week 3 to week 5 of ex vivo culture. Groups compared by student's t-test, *=p<0.05.

FIGS. 18A-I: The antitumor potency of CAL-101 primed T cells is CD62L and CD127 independent. (A) Sort diagram with post-sort analysis of CD62L and CD44 expression on pmel-1 T cells. (B) Average tumor burden (mm²) and (C) percent survival of mice with B16F10 were treated with 1×10⁶ bulk vehicle, CD62L⁺ vehicle, naïve or CAL-101 pmel-1 cells compared to no treatment; n=5-10 mice/group/experiment, representative of 2 independent experiments. Kaplan Meier curve analyzed by log rank test; *=p<0.05. (D) Frequency of donor CAL-101 T cells in mice receiving isotype or IL-7 depleting antibody; n=5 mice/group. One way repeated measures ANOVA; ns=not statistically significant. (See also FIG. 21). (E) Average tumor burden and (F) percent survival of of isotype or IL-7 depleted mice receiving CAL-101 primed donor cells; n=10 mice/group/experiment, representative of 2 independent experiments. Kaplan Meier curve analyzed by log rank test; ns=not statistically significant. (G) Percent specific lysis of hgp100 loaded splenocytes by CAL-101 primed pmel-1 T cells in isotype treated or IL-7 depleted mice 53 days post-transfer; n=6-7 mice/group from one experiment. Comparison by student's t-test; ns=not statistically significant. (H) Average tumor burden and (I) percent survival of mice receiving CAL-101 treated donor cells with IL-2 complex, without IL-2 complex plus isotype, or without IL-2 complex plus IL-7 depletion compared to no treatment; n=7-9 mice/group in one experiment. Kaplan Meier curve analyzed by log rank test; *=p<0.05.

FIGS. 19A-C: CAL-101 T cells share transcriptional characteristics with stem memory T cells. (A) Differential expression of memory and effector associated genes in CAL-101 or AKTi treated human T cells compared to vehicle analyzed using RNA sequencing (See also FIG. 22); n=3 normal donors. (B) Western blot of nuclear protein extracts from vehicle, AKTi, or CAL-101 treated human T cells, B=β-catenin, L=Lef1, T=Tcf7, H=Histone H3; representative of 4-6 donors. (C) Quantified protein levels relative to Histone H3, RROD GOI/H=relative ratio of optical density (gene of interest over Histone H3); n=4-6 normal donors. One way repeated measures ANOVA; ns=not statistically significant, *=p<0.05.

FIGS. 20A-C: Depletion of IL-7 does not alter numbers or memory phenotype of donor CAL-101 T cells. (A) Individual tumor burden (mm²) and (B) percent survival of mice which received no T cell treatment, or 8×10⁵ pmel-1 CD8⁺ T cells primed with vehicle, 1 or 10 μM AKTi or CAL-101; n=10-12 mice/group. Kaplan Meier curve analyzed by log rank test; ns=not statistically significant, *=p<0.05, ****=p<0.0001. (C) Growth of human peripheral T cells primed with vehicle (DMSO), 1 or 10 μM AKTi, and 1 or 10 μM CAL-101 8 days following antigen stimulation; n=3 independent cultures. One way repeated measures ANOVA; *=p<0.05.

FIGS. 21A-C: Depletion of IL-7 does not alter numbers or memory phenotype of donor CAL-101 T cells. (A) Analysis of in vivo numbers of transferred pmel-1 T cells primed with vehicle, AKTi, or CAL-101 10 days after transfer into mice receiving isotype or IL-7 depleting antibody, n=5 mice/group from one experiment. One way repeated measures ANOVA; ns=not statistically significant, **=p<0.01, ***=p<0.001. (B-C) Memory phenotype of transferred pmel-1 T cells primed with vehicle, AKTi, or CAL-101 10 days after transfer into (B) isotype or (C) IL-7 depleted mice; n=5 mice/group from one experiment. One way repeated measures ANOVA; ns=not statistically significant, *=p<0.05, **=p<0.01, ****=p<0.0001.

FIG. 22: Differential expression of genes in CAL-101 or AKTi treated T cells. Differential expression of genes hypothesized to be altered in expression (Genes of Interest) as well as genes discovered to be markedly altered (Discovered Gene Alterations) in CAL-101 or AKTi treated cells compared to vehicle using RNA sequencing; n=3 normal donors.

FIG. 23: CAL-101 pmel-1 persists, resists senescence and is functional. 30 days post ACT, mice infused with CAL-101, Ly294004 or AKTi-pmel-1 were tracked by A) Thy1.1⁺ in tumor-draining lymphnodes and lung, and B) % IFN-γ, IL-2, perforin & KLRG1 monitored after B16 recognition, ICC stain. N=4 mice/grp. *P<0.05 & **P<0.01. Student t-test.

FIG. 24: Pharmaceutical not genetic PI3Kδ blockade mediates durable memory T cell responses to self and tumor. A) Vitiligo incidence (d35) & B) 38 d post transfer, surviving mice previously treated with CAL-101- or PI3Kδ^{-/-}-pmel-1 & IL-2cx was re-challenged with a subQ B16 tumor. N=6-10 mice/grp. CAL>PI3Kδ^{-/-}, vitiligo *P=0.023. Student t-test, tumor growth **P<0.01, Log rank analysis.

FIG. 25. CAL-101 pmel-1 has high mitochondrial SRC and low Awm relative to PI3Kδ^{-/-}pmel-1. A) Percent change in 02 consumption rates (OCR) in real time under basal conditions & in response to respiratory inhibitors after FCCP injection via seahorse & B) mitochondrial membrane potential (Δψm) measured TMRM by flow. Rep of 2-9 exp. *P<0.05&**P<0.001. Student t-test.

FIG. 26: CAL-101 enhances TIL. TILs expanded from cancer patients secrete more IFN-γ, less KLRG-1 & express CD62L when CAL-101 treated. TILs expanded w/OTK3, IL-2 & CAL-101 (10 μm) 2 wks by flow. N=4.

FIG. 27: TIL expand robustly with CAL-101 and sustain CD62L 30 days post REP. TILs were activated with high dose IL-2 and then underwent REP expansion with feeder cells and OKT3 every 10 days. Expansion and CD62L analyzed overtime with flow cytometry.

### DESCRIPTION OF ILLUSTRATIVE EMBODIMENTS

The present disclosure overcomes challenges associated with current technologies by providing methods for the production and use of modified T cells. ICOS co-stimulation enhances the antitumor activity of adoptively transferred Th17 cells in mice with large tumors. The present studies found that ICOS activation increased RORγt and cMaf expression as well as IL-17 and IFN-γ secretion by Th17 cells. Moreover, ICOS induced PI3K/p110δ/Akt and Wnt/β-catenin signaling pathways, which were diminished in ICOS−/− Th17 cells. Pharmacological inhibition of p110γ and β-catenin impaired the function of Th17 cells (using CAL-101 and indomethacin, respectively). Unexpectedly, however, ICOS-activated Th17 cells elicited an even better antitumor immunity in vivo when they were in vitro inhibited of both pathways. PI3K/p110δ inhibition supported the generation of Th17 cells with a central memory phenotype that expressed nominal FoxP3 and heighted Tcf7 expression. In addition, reversible inhibition of β-catenin enhanced the multi-functionality and engraftment of Th17 cells. The present data demonstrate that small molecules, already approved for human use, can be exploited to enhance cancer immunotherapy. Thus, the present disclosure provides a new human T cell subset with remarkable antitumor properties which can be harnessed to design next generation cancer immunotherapies for the clinic.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of enhanced Th17 cells or other immune cell therapy.

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The term “immune checkpoint” refers to a molecule such as a protein in the immune system which provides inhibitory signals to its components in order to balance immune reactions. Known immune checkpoint proteins comprise CTLA-4, PD1 and its ligands PD-L1 and PD-L2 and in addition LAG-3, BTLA, B7H3, B7H4, TIM3, MR or any other immune receptor that inhibits the proliferation and activation of immune cells. The pathways involving LAG3, BTLA, B7H3, B7H4, TIM3, and MR are recognized in the art to constitute immune checkpoint pathways similar to the CTLA-4 and PD-1 dependent pathways (see e.g. Pardoll, 2012; Mellman et al., 2011).

An “immune checkpoint inhibitor” refers to any compound inhibiting the function of an immune checkpoint protein. Inhibition includes reduction of function and full blockade. In particular the immune checkpoint protein is a human immune checkpoint protein. Thus the immune checkpoint protein inhibitor in particular is an inhibitor of a human immune checkpoint protein.

“Long-term engraftment” is defined herein as the stable transplantation of cells provided by the methods herein into a recipient such that the transplanted cells persist in the host blood and/or bone marrow more than 10 weeks, preferably more than 20 weeks. In addition, long-term engraftment can be characterized by the persistence of transplantation cells in serially transplanted mice.

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. In some embodiments, CARs comprise an intracellular activation domain, a transmembrane domain, and an extracellular domain comprising a tumor associated antigen binding region. In particular aspects, CARs comprise fusions of single-chain variable fragments (scFv) derived from monoclonal antibodies, fused to CD3-zeta a transmembrane domain and endodomain. The specificity of other CAR designs may be derived from ligands of receptors (e.g., peptides) or from pattern-recognition receptors, such as Dectins. In certain cases, the spacing of the antigen-recognition domain can be modified to reduce activation-induced cell death. In certain cases, CARs comprise domains for additional co-stimulatory signaling, such as CD3ζ, FcR, CD26, CD27, CD28, CD137, DAP10, ICOS, 41BB, OX40 and or any other molecule. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

As used herein, the term “antigen” is a molecule capable of being bound by an antibody or T-cell receptor. An antigen may generally be used to induce a humoral immune response and/or a cellular immune response leading to the production of B and/or T lymphocytes.

The terms “tumor-associated antigen,” “tumor antigen” and “cancer cell antigen” are used interchangeably herein. In each case, the terms refer to proteins, glycoproteins or carbohydrates that are specifically or preferentially expressed by cancer cells.

### II. TH17 CELLS

Embodiments of the present disclosure concern obtaining and administering enhanced ICOS-stimulated Th17 cells to a subject as an immunotherapy to target cancer cells. Several basic approaches for the derivation, activation and expansion of functional anti-tumor effector T cells have been described in the last two decades. These include: autologous cells, such as tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization which can be used in the methods of the present disclosure.

A. T Cell Preparation

In some embodiments, the T cells are derived from the blood, bone marrow, lymph, or lymphoid organs. In some aspects, the cells are human cells. In certain embodiments, T cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art. The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells, Innate lymphocyte cells or other immune cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells). In one embodiment, the cells (e.g., CD8⁺ cells or CD3⁺ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, any other immune markers and/or IL7-Ra (CD127). In some examples, CD8⁺ T cells are enriched for cells positive for CD45RO (or negative for CD45RA) and for CD62L.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (TC_{M}) cells is carried out to increase efficacy, such as to improve long-term survival, expansion, and/or engraftment following administration, which in some aspects is particularly robust in such sub-populations. See Terakuraet et al., 2012; Wang et al., 2012. In some embodiments, combining T_{CM}-enriched CD8⁺ T cells and CD4⁺ T cells further enhances efficacy.

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days. For example, the cells may be cultured from 5 days, 5.5 days, or 5.8 days to 21 days, 21.5 days, or 21.8 days, such as from 10 days, 10.5 days, or 10.8 days to 14 days, 14.5 days, or 14.8 days.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days, preferably about 14 days. More preferably, rapid expansion provides an increase of at least about 200-fold (e.g., 200-, 300-, 400-, 500-, 600-, 700-, 800-, 900-, or greater) over a period of about 10 to about 14 days, preferably about 14 days.

Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being preferred. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A2 (HLA-A2) binding peptide, in the presence of a T-cell growth factor, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example.

In particular aspects, the Th17 cells are activated by costimulation with inducible coactivator (ICOS). In some aspects, the ICOS stimulation is performed by culturing the Th17 cells with beads, such as magnetic beads, coated with anti-ICOS with or without anti-CD3 beads. The cell expansion can be performed in the presence cytokines, such as IL-2. The ratio of beads to T cells may be in the range of 1:1 to 1:50, such as 1:5 to 1:25, particularly such as 1:10.

The autologous T-cells can be modified to express a T-cell growth factor that promotes the growth and activation of the autologous T-cells. Suitable T-cell growth factors include, for example, interleukin (IL)-2, IL-7, IL-15, IL-12 and or other cytokines or small molecule drugs (such as PI3 kinase or AKT inhibitors, etc.). Suitable methods of modification are known in the art. See, for instance, Sambrook et al., 2001 and Ausubel et al., 1994. In particular aspects, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression.

B. Genetically Engineered Antigen Receptors

The T cells can be genetically engineered to express antigen receptors such as engineered TCRs and/or chimeric antigen receptors (CARs). For example, the autologous T-cells are modified to express a T cell receptor (TCR) having antigenic specificity for a cancer antigen. Suitable TCRs include, for example, those with antigenic specificity for a mesothelin antigen. Suitable methods of modification are known in the art. See, for instance, Sambrook and Ausubel, supra. For example, the T cells may be transduced to express a T cell receptor (TCR) having antigenic specificity for a cancer antigen using transduction techniques described in Heemskerk et al., 2008 and Johnson et al., 2009.

In some embodiments, the T cells comprise one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

In some embodiments, the CAR contains an extracellular antigen-recognition domain that specifically binds to an antigen. In some embodiments, the antigen is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., Curr. Opin. Immunol., 2012; Wu et al., 2012. In some aspects, the genetically engineered antigen receptors include a CAR as described in U.S. Pat. No. 7,446,190, and those described in International Patent Application Publication No.: WO/2014055668 A1.

In some aspects, the tumor antigen is a human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53 or cyclin (Dl). For example, the target antigen is hTERT or survivin. In some aspects, the target antigen is CD38. In other aspects, the target antigen is CD33 or TIM-3. In other aspects, it is CD26, CD30, CD53, CD92, CD148, CD150, CD200, CD261, CD262, or CD362. In some embodiments, the engineered immune cells can contain an antigen that targets one or more other antigens. In some embodiments, the one or more other antigens is a tumor antigen or cancer marker. Other antigens include orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, Ll-cell adhesion molecule, MAGE-A1, mesothelin, MUC1, MUC16, PSCA, NKG2D Ligands, NY-ESO-1, MART-1, gplOO, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD 123, CS-1, c-Met, GD-2, and MAGE A3, CE7, Wilms Tumor 1 (WT-1), a cyclin, such as cyclin Ak (CCNA1), and/or biotinylated molecules, and/or molecules expressed by HIV, HCV, HBV or other pathogens. In particular aspects, the tumor antigen is mesothelin.

1. Chimeric Antigen Receptors

In certain embodiments, the T cells are genetically modified to express a chimeric antigen receptor. In some embodiments, the chimeric antigen receptor comprises: a) an intracellular signaling domain, b) a transmembrane domain, and c) an extracellular domain comprising an antigen binding region.

In some embodiments, the engineered antigen receptors include chimeric antigen receptors (CARs), including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb). The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD5, CD9, CD 16, CD22, CD33, CD37, CD64, CD80, CD86, CD 134, CD137, CD 154 or any other molecule. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

The CAR generally includes at least one intracellular signaling component or components. In some embodiments, the CAR includes an intracellular component of the TCR complex, such as a TCR CD3⁺ chain that mediates T-cell activation and cytotoxicity, e.g., CD3 zeta chain. Thus, in some aspects, the antigen binding molecule is linked to one or more cell signaling modules. In some embodiments, cell signaling modules include CD3 transmembrane domain, CD3 intracellular signaling domains, and/or other CD transmembrane domains. In some embodiments, the CAR further includes a portion of one or more additional molecules such as Fc receptor γ, CD8, CD4, CD25, or CD16. For example, in some aspects, the CAR includes a chimeric molecule between CD3-zeta (CD3-Q or Fc receptor γ and CD8, CD4, CD25 or CD16. In specific embodiments, intracellular receptor signaling domains in the CAR include those of the T-cell antigen receptor complex, such as the zeta chain of CD3, also Fcγ RIII costimulatory signaling domains, CD28, CD27, DAP10, CD137, OX40, CD2, alone or in a series with CD3zeta, for example. In specific embodiments, the intracellular domain (which may be referred to as the cytoplasmic domain) comprises part or all of one or more of TCR zeta chain, CD28, CD27, OX40/CD134, 4-1BB/CD137, FcεRIγ, ICOS/CD278, IL-2Rbeta/CD122, IL-2Ralpha/CD132, DAP10, DAP12, and CD40. In some embodiments, one employs any part of the endogenous T-cell receptor complex in the intracellular domain. One or multiple cytoplasmic domains may be employed, as so-called third generation CARs have at least two or three signaling domains fused together for additive or synergistic effect, for example.

It is contemplated that the chimeric construct can be introduced into T cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into T cells. Suitable vectors for use in accordance with the method of the present invention are non-replicating in the T cells. A large number of vectors are known that are based on viruses, where the copy number of the virus maintained in the cell is low enough to maintain the viability of the cell, such as, for example, vectors based on HIV, SV40, EBV, HSV, or BPV.

2. T Cell Receptor (TCR)

In some embodiments, the genetically engineered antigen receptors include recombinant T cell receptors (TCRs) and/or TCRs cloned from naturally occurring T cells. A “T cell receptor” or “TCR” refers to a molecule that contains a variable a and β chains (also known as TCRa and TCRp, respectively) or a variable γ and δ chains (also known as TCRγ and TCR5, respectively) and that is capable of specifically binding to an antigen peptide bound to a MHC receptor. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MEIC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; see also Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the (3-chain can contain a further hypervariability (HV4) region.

In some embodiments, the TCR chains contain a constant domain. For example, like immunoglobulins, the extracellular portion of TCR chains (e.g., a-chain, β-chain) can contain two immunoglobulin domains, a variable domain (e.g., Vₐ or Vp; typically amino acids 1 to 116 based on Kabat numbering Kabat et al., 1991) at the N-terminus, and one constant domain (e.g., a-chain constant domain or Cₐ, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3y chain, a CD35 chain, two CD3s chains, and a homodimer of CD3 chains. The CD3y, CD35, and CD3s chains are highly related cell surface proteins of the immunoglobulin superfamily containing a single immunoglobulin domain. The transmembrane regions of the CD3y, CD35, and CD3s chains are negatively charged, which is a characteristic that allows these chains to associate with the positively charged T cell receptor chains. The intracellular tails of the CD3y, CD35, and CD3s chains each contain a single conserved motif known as an immunoreceptor tyrosine-based activation motif or ITAM, whereas each CD3 chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains a and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. [0140] In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, a high-affinity T cell clone can be isolated from a patient, and the TCR isolated. In some embodiments, the T-cells can be a cultured T-cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005. In some embodiments, phage display is used to isolate TCRs against a target antigen (see, e.g., Varela-Rohena et al., 2008 and Li, 2005. In some embodiments, the TCR or antigen-binding portion thereof can be synthetically generated from knowledge of the sequence of the TCR.

3. Antigen-Presenting Cells

Antigen-presenting cells, which include macrophages, B lymphocytes, and dendritic cells, are distinguished by their expression of a particular MHC molecule. APCs internalize antigen and re-express a part of that antigen, together with the MHC molecule on their outer cell membrane. The major histocompatibility complex (MHC) is a large genetic complex with multiple loci. The MHC loci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans the MHC is referred to as the HLA complex and in mice the H-2 complex.

In some cases, aAPCs are useful in preparing therapeutic compositions and cell therapy products of the embodiments. For general guidance regarding the preparation and use of antigen-presenting systems, see, e.g., U.S. Pat. Nos. 6,225,042, 6,355,479, 6,362,001 and 6,790,662; U.S. Patent Application Publication Nos. 2009/0017000 and 2009/0004142; and International Publication No. WO2007/103009.

aAPC systems may comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD86, CD64 (FcγRI), 41BB ligand, and IL-21. Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), which promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001.

In some embodiments, the presently disclosed process can be used to genetically modify T cells derived from peripheral blood and/or umbilical cord blood to express CAR(s) that can be numerically expanded in vitro using aAPC (Singh et al., 2008; Singh et al., 2011; Shah et al., 2013). The process has implications for cell and gene therapy, due to the relative ease of DNA plasmid production, electroporation, use of thawed γ-irradiated master-bank aAPC, and can be readily transferred to facilities operating in compliance with current good manufacturing practice (cGMP) for clinical trials.

In one embodiment, aAPCs are also subjected to a freeze-thaw cycle. In an exemplary freeze-thaw cycle, the aAPCs may be frozen by contacting a suitable receptacle containing the aAPCs with an appropriate amount of liquid nitrogen, solid carbon dioxide (i.e., dry ice), or similar low-temperature material, such that freezing occurs rapidly. The frozen aAPCs are then thawed, either by removal of the aAPCs from the low-temperature material and exposure to ambient room temperature conditions, or by a facilitated thawing process in which a lukewarm water bath or warm hand is employed to facilitate a shorter thawing time. Additionally, aAPCs may be frozen and stored for an extended period of time prior to thawing. Frozen aAPCs may also be thawed and then lyophilized before further use. Preferably, preservatives that might detrimentally impact the freeze-thaw procedures, such as dimethyl sulfoxide (DMSO), polyethylene glycols (PEGs), and other preservatives, are absent from media containing aAPCs that undergo the freeze-thaw cycle, or are essentially removed, such as by transfer of aAPCs to media that is essentially devoid of such preservatives.

### III. METHODS OF TREATMENT

Provided herein are methods for treating or delaying progression of cancer in an individual comprising administering to the individual an effective amount an enhanced ICOS-stimulated Th17 cell therapy. Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer.

In some embodiments, the individual has cancer that is resistant (has been demonstrated to be resistant) to one or more anti-cancer therapies. In some embodiments, resistance to anti-cancer therapy includes recurrence of cancer or refractory cancer. Recurrence may refer to the reappearance of cancer, in the original site or a new site, after treatment. In some embodiments, resistance to anti-cancer therapy includes progression of the cancer during treatment with the anti-cancer therapy. In some embodiments, the cancer is at early stage or at late stage.

In some embodiments of the methods of the present disclosure, activated CD4 and/or CD8 T cells in the individual are characterized by γ-IFN producing CD4 and/or CD8 T cells and/or enhanced cytolytic activity relative to prior to the administration of the combination. γ-IFN may be measured by any means known in the art, including, e.g., intracellular cytokine staining (ICS) involving cell fixation, permeabilization, and staining with an antibody against γ-IFN. Cytolytic activity may be measured by any means known in the art, e.g., using a cell killing assay with mixed effector and target cells.

In some aspects, the T cells are administered in combination with at least one additional anti-cancer therapy. The T cell therapy may be administered before, during, after, or in various combinations relative to an anti-cancer agent. The administrations may be in intervals ranging from concurrently to minutes to days to weeks. In embodiments where the T cell therapy is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the T therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In some embodiments, the subject can be administered nonmyeloablative lymphodepleting chemotherapy prior to the T cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, a T-cell growth factor that promotes the growth and activation of the autologous T cells is administered to the subject either concomitantly with the autologous T cells or subsequently to the autologous T cells. The T-cell growth factor can be any suitable growth factor that promotes the growth and activation of the autologous T-cells. Examples of suitable T-cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2. IL-12 is a preferred T-cell growth factor.

The T cell therapy and anti-cancer agent may be administered by the same route of administration or by different routes of administration. In some embodiments, the T cell therapy and/or anti-cancer agent is administered intravenously, intramuscularly, subcutaneously, topically, orally, transdermally, intraperitoneally, intraorbitally, by implantation, by inhalation, intrathecally, intraventricularly, or intranasally. An effective amount of the T cell therapy and anti-cancer agent may be administered for prevention or treatment of disease. The appropriate dosage of the T cell therapy and anti-cancer agent be determined based on the type of disease to be treated, severity and course of the disease, the clinical condition of the individual, the individual's clinical history and response to the treatment, and the discretion of the attending physician.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

A. Pharmaceutical Compositions

Also provided herein are pharmaceutical compositions and formulations comprising the enhanced ICOS-stimulated Th17 cell therapy, optionally an anti-cancer agent and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22nd edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

B. Anti-Cancer Therapy

In certain embodiments, the compositions and methods of the present embodiments involve an enhanced ICOS-stimulated Th17 cell therapy in combination with at least additional anti-cancer agent. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may another subset of T cells, such as CD8⁺ T cells. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

Various combinations may be employed. For the example below an enhanced Th17 cell therapy is “A” and an anti-cancer therapy is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegaIl); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen (Carter et al., 2008; Teicher 2014; Leal et al., 2014). Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach (Teicher 2009) and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons a, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. 20140294898, 2014022021, and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; Camacho et al., 2004; Mokyr et al., 1998 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WOO 1/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. U.S. Pat. Nos. 5,844,905, 5,885,796 and International Patent Application Nos. WO1995001994 and WO1998042752; all incorporated herein by reference, and immunoadhesions such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IV. ARTICLES OF MANUFACTURE OR KITS

An article of manufacture or a kit is provided comprising enhanced ICOS-stimulated Th17 cells is also provided herein. The article of manufacture or kit can further comprise a package insert comprising instructions for using the enhanced Th17 cells to treat or delay progression of cancer in an individual or to enhance immune function of an individual having cancer. Any of the T cells described herein may be included in the article of manufacture or kits. In some embodiments, the T cells are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Pharmaceutically Modified T Cells

ICOS Signaling Augments the Antitumor Activity of Th17 Cells:

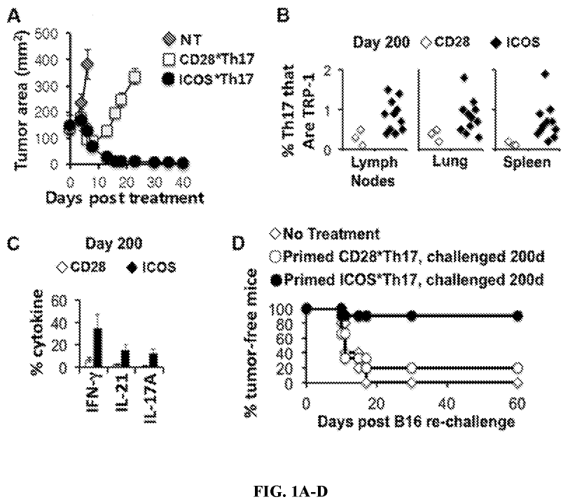

Muranski et al. reported that murine Th17 cells regress melanoma superior to Th1 cells (1-3). It was found that human Th17 cells stimulated with ICOS, but not CD28, possessed potent antitumor activity and persist in xenogeneic mice bearing human mesothelioma (9). Herein, these findings were recapitulated in a syngeneic mouse model of melanoma using transgenic TRP-1 CD4+ T cells programmed towards a Th17 phenotype and expanded with α-CD3-beads coated with either CD28 or ICOS agonist. These transgenic mice have a MHC-II restricted TCR on their CD4+ T cells that recognizes a tyrosinase-related protein 1 (trp-1), expressed on normal melanocytes and melanoma. ICOS co-stimulation significantly improved the antitumor activity of TRP-1 Th17 cells (FIG. 1A) compared to CD28 signal. ICOS, but not CD28 co-stimulation, promoted long-lived memory Th17 cells, present 200 days post-transfer. These cells were detected in higher frequency via their TCR V1314 expression in lymph nodes, lung and spleen (FIG. 1B) and produced effector cytokines IFN-γ, IL-21, IL-17A, upon ex vivo B16F10 cancer cells activation (FIG. 1C). In contrast to CD28 co-stimulation, Th17 lymphocytes generated with ICOS mediated long-term tumor protection against B16F10 tumor re-challenge 200 days post-transfer (FIG. 1D). Our data indicate that ICOS signaling promotes the generation of durable memory Th17 cells with potent antitumor immunity.

ICOS Induces Wnt/β-Catenin and PI3K/p110δ Signaling Pathways in Th17 Cells:

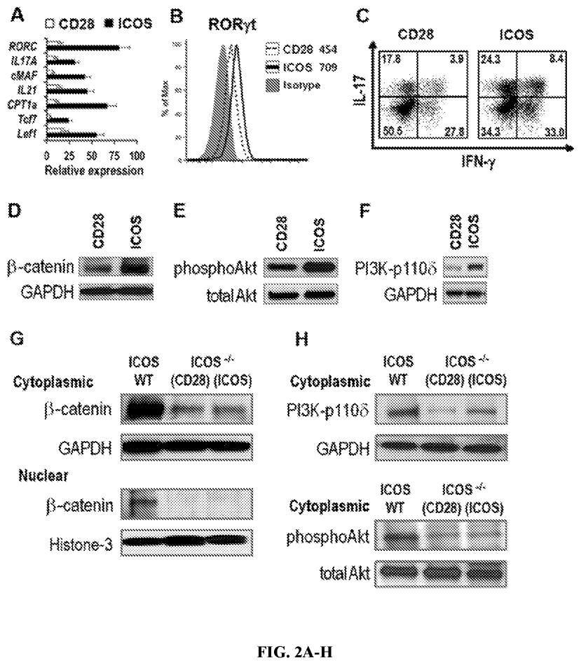

To uncover the mechanisms underlying how ICOS signaling enhanced Th17 antitumor activity, the gene expression profiles of ICOS vs. CD28 co-stimulated Th17 cells were compared using Real-time PCR analysis. ICOS signaling induced several genes that might be responsible for bolstering their function and memory response (FIG. 2A). Primarily, ICOS increased the expression of IL-17A and Rorc, a master transcription factor for Th17 cells differentiation (FIG. 2A). It was confirmed enhanced RORγt expression by flow cytometry (FIG. 2B). Subsequently the percent of cells producing IL-17 and IFNγ was higher after ICOS co-stimulation (FIG. 2C). Secondly, ICOS signaling enhanced Cmaf, a Th17 cells-associated transcription factor that triggers IL-21 production (21). IL-21 gene expression was also increased (FIG. 2A). Furthermore, ICOS induced Cpt1a, a mitochondrial gene that plays a role in supporting CD8⁺ T cell memory (22).

Interestingly, ICOS activation induced to greater extent than CD28 signal the expression of Lef1 and Tcf7 in Th17 cells (FIG. 2A). Genes involved in the Wnt/β-catenin pathway, which are expressed in HSCs, stem cell-like CD8⁺ T cells and Th17 cells (3). Lef1 and Tcf7 are transcription factors, which mediate a nuclear response to Wnt signals by interacting with β-catenin (23). Therefore, the expression of β-catenin was determined by Western blot analysis in Th17 cells co-stimulated with ICOS vs. CD28. β-catenin was induced to the greater amount by ICOS signaling and remained high upon re-stimulation (FIG. 2D). Moreover, ICOS increased phosphoAkt expression and induced PI3K-p110δ in Th17 cells compared to CD28 co-stimulation (FIG. 2E-F).

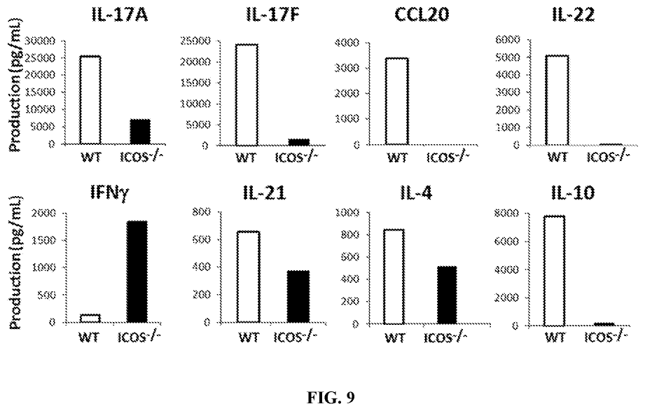

To confirm the role of ICOS on regulating PI3K/Akt and Wnt/β-catenin pathways, Th17 cells were generated from ICOS^{-/-} TRP-1 Tg mice. These cells lack of ICOS but retain CD28 expression (FIG. 8A). Thus, they were expanded in vitro with TRP-1 peptide and either an ICOS or CD28 agonist. Although the majority of Th17 cells were activated, as indicated by comparable CD69 expression (FIG. 8B), ICOS^{-/-} Th17 did not grow as well as WT Th17 cells. Western blot analysis (day 8) revealed that β-catenin, p110δ and phosphoAkt were expressed to a lesser extent in ICOS^{-/-} Th17 cells (FIG. 2G, H). Importantly, the expression of these proteins could not be induced using CD28 agonist, underscoring the importance of ICOS signaling. These results were confirmed using a flow cytometry. Moreover, compared to WT Th17 cells, ICOS^{-/-} Th17 cells produced less inflammatory cytokines: IL-17A, IL-17F, IL-22, CCL20, IL-21, IL-4 and IL-10 (FIGS. 8C and 9). ICOS^{-/-} Th17 secreted more IFN-γ than WT Th17 cells (FIG. 9). These data indicate that ICOS induces of Wnt/β-catenin and PI3K/Akt pathways in Th17 cells, yet how these individual pathways contribute to the function, persistence and antitumor activity of ICOS stimulated Th17 cells remains unclear.

Pharmacological Inhibition of p110δ and/or β-Catenin Alters Th17 Function:

Given that induction of both PI3K and Wnt pathways are necessary for HSCs self-renewal and expansion (7), it was hypothesized that ICOS signaling supports durable memory Th17 cells via the same mechanism. Therefore, we pharmacologically suppressed in vitro 1) PI3K/Akt pathway using CAL-101, a highly-specific p110δ subunit small molecule inhibitor (24) or Ly294002, a pan inhibitor of PI3Ks activity (25) and/or 2) Wnt/β-catenin pathway using indomethacin (Indo), a COX2 inhibitor shown to suppress β-catenin expression (26,27).

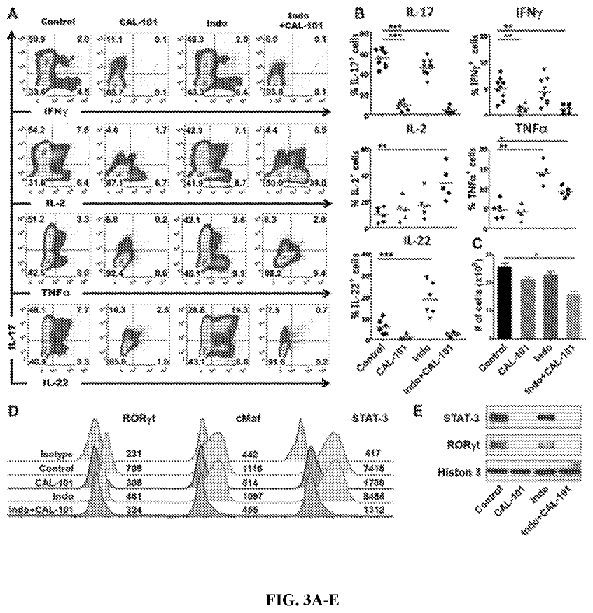

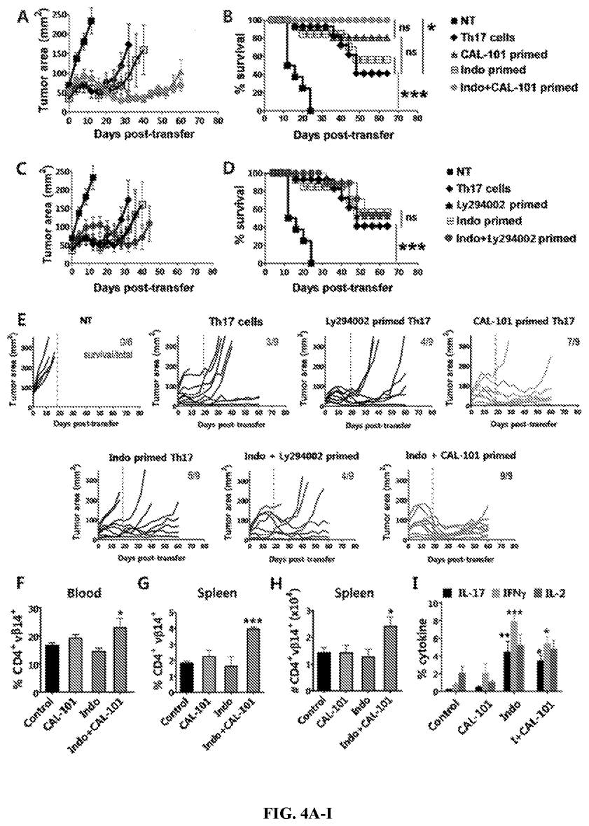

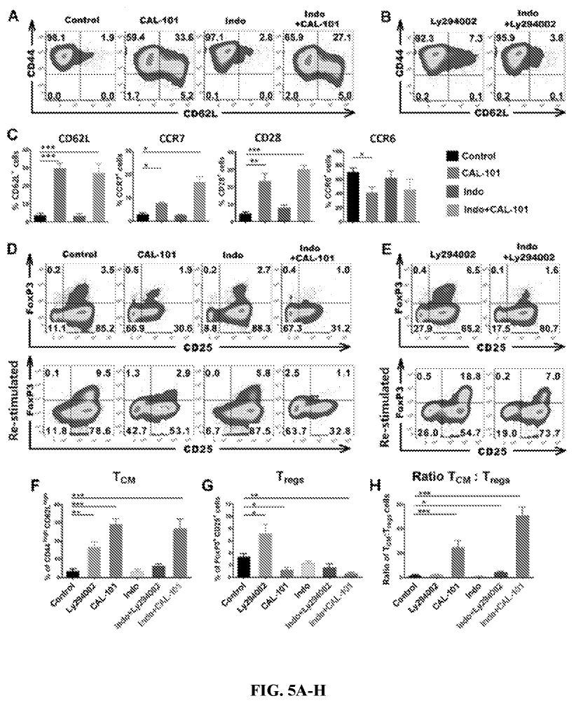

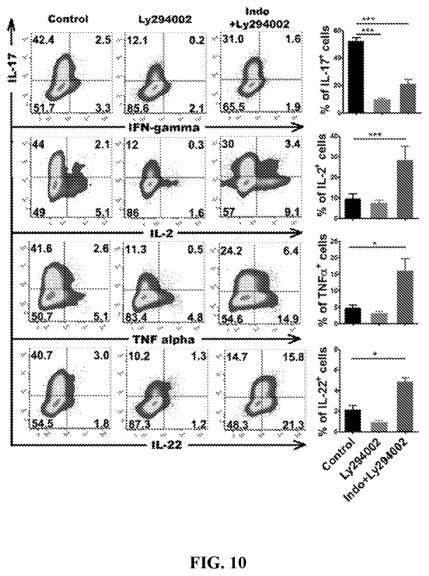

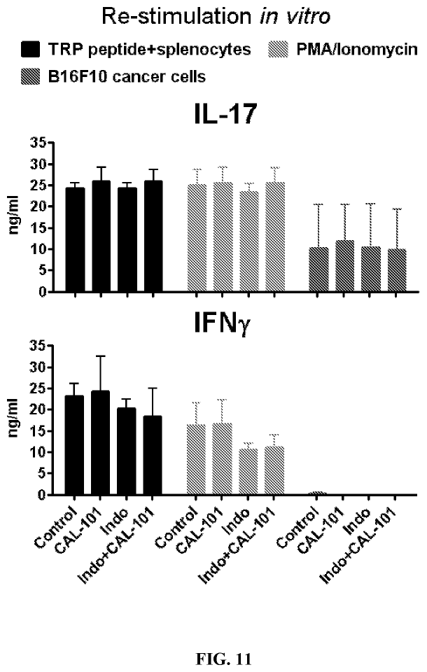

The percentage of Th17 cells able to secrete various cytokines after 8 days of expansion in vitro in the presence of these small molecules, as indicated (FIG. 3A, B). It was found that inhibition of p110δ using CAL-101 reduced the ability of Th17 cells to secrete IL-17A and IFN-γ, but had no impact on IL-2, IL-22 or TNF-α production compared to un-treated Th17 cells (FIG. 3A, B). Three days after priming, CAL-101 treatment decreased the ability of Th17 cells to secrete of IL-17A, IFN-γ, IL-22, CCL-20, IL-2, IL-9 and GM-CSF, but had less effect their ability to secrete IL-10 or IL-21 (FIG. 10). Similarly results were obtained using Ly294002 (FIG. 11).

Inhibition of β-catenin using Indo did not impact the ability of ICOS-stimulated Th17 cells to secrete IL-17 or IFNγ, but increased their capacity to secrete IL-22 and TNF-α (FIG. 3A, B). Although Kang et al. showed that inhibition of the Wnt/β-catenin pathway via blocking the Frizzled receptor enhanced IL-17A production by human Th17 cells (28), this was not observed in murine Th17 after suppressing β-catenin by Indo. In contrast, on day 3, IL-17A as well as IFN-γ, CCL-20 and IL-9 secretion decreased in the presence of Indo (FIG. 10). Noteworthy, there was no difference in IL-2, IL-10, IL-21 or GM-CSF secretion after Indo treatment compared to control Th17 cells (FIG. 10).

Both ICOS-induced pathways were also inhibited simultaneously, which abolished IL-17A and IFNγ production, as well as secretion of IL-22, CLL20, IL-9 and GM-CSF by ICOS-activated Th17 cells (FIG. 3A, B and FIG. 10). IL-10 and IL-4 production was not altered (FIG. 10). Interestingly, it was found that treating ICOS-stimulated Th17 cells in vitro with both inhibitors significantly elevated their capacity to secrete TNF-α and IL-2 (FIG. 3A, B). A similar pattern was observed using Indo and Ly29004 (FIG. 11). Although Th17 cell growth was slightly disrupted and the amount of late apoptotic cells (PrAnnexinV⁺) was transiently increased when both pathways were suppressed, these cells still expanded effectively in the presence of both drugs in vitro (FIG. 3C). Treatment with either CAL-101 alone or Indo alone did not impair the growth or survival of ICOS-stimulated Th17 cells (FIG. 3C).

Alterations of cytokine production by Th17 cells were associated with decreased transcription factors: RORγt, cMaf and STAT-3 (FIG. 3D). In vitro treatment of ICOS-activated Th17 cells with CAL-101 alone or Indo plus CAL-101 decreased RORγt expression from 65.5±9.5% to 21.9±8.1% and 17.4±5.3%, respectively (p<0.01). Similarly, cMaf decreased from 62.9±4.8% to 42.5±3.9% and 35.7±5.1%, after treatment with CAL-101 or Indo plus CAL-101, respectively (p<0.05). Finally, STAT-3 expression was inhibited almost completely from approximately 83% expression at control conditions to 13.8±5.2% after CAL-101 treatment (p<0.001) and 10.8±2.1% after Indo plus CAL-101 treatment (p<0.001). We confirmed our findings that CAL-101 and/or Indo compromise nuclear RORγt and STAT3 expression by Western blot analysis (FIG. 3D). These data revealed that Th17 cells expanded in vitro with inhibitors of ICOS-induced pathways were less functional, thus it was suspected that those cells transferred to tumor-bearing mouse would be compromised in their ability to treat melanoma.

ICOS Stimulated Th17 Cells Mediate Superior Antitumor Response after Priming In Vitro with β-Catenin and p110δ Inhibitors:

Based on the findings that pharmacological inhibition of Wnt/β-catenin and PI3K/p110δ pathways impair the function of ICOS-stimulated Th17 cells, it was hypothesized that those cells would have diminished effectiveness after adoptive transfer. To test this idea, C57BL/6 mice were inoculated with melanoma B16F10 cancer cells and let the tumor grow for 10-12 days: manifesting a large established, poorly immunogenic melanoma tumor, highly relevant to patients with stage IV metastatic malignancy. One day before ACT, a sub-lethal dose (5Gy) of total body irradiation (TBI) was administered as a host preconditioning regimen, that bolsters treatment outcome in mice and humans (29). Fewer TRP-1 ICOS stimulated Th17 cells (0.75×10⁶) were used, which do not cure the mouse (tumor relapses 21-30 days post-transfer) to create a treatment window to determine how priming with CAL-101 and/or Indo regulates their antitumor activity.

Surprisingly and against the hypothesis, ICOS co-stimulated Th17 cells expanded in vitro in the presence of Indo plus CAL-101 mediating superior tumor regression compared to un-treated Th17 cells (FIG. 4A), as well as enhancing their overall survival (FIG. 4B). Th17 cells primed just with CAL-101 mediated almost as potent tumor regression as cells primed with both inhibitors (Indo+CAL-101) (FIG. 4A), but did not significantly augment survival time of mice compared to control ICOS-stimulated Th17 cells (referred as “Th17 cells” in the figures) (FIG. 4B). Only when Th17 cells were primed with Indo plus CAL-101 all mice (n=9) survived over 64 days. Priming Th17 cells with only Indo did not significantly improve their antitumor efficiency or survival upon ACT, compared to control Th17 cells. Note that CAL-101 could not simply be replaced with Ly294002 (pan PI3K inhibitor) to bolster the therapeutic index of Th17 cells treated with Indo (Th17 cells Indo+CAL-101 primed>Th17 cells Indo+Ly249002 primed). In other words, Th17 cells activated with ICOS and cultured in the presence of both CAL-101 and Indo regressed tumor and extended survival of mice to a far greater extent than when treated with Ly294002 and Indo (compare FIG. 4A, B to FIG. 4C, D). Moreover, it should be appreciated that while CAL-101 alone enhanced that antitumor activity of Th17 cells, Ly294002 alone was unable to impact treatment outcome by Th17 cells. As in FIG. 4E, the tumor growth curve of individual mice is display for all 7 different groups.

Next, the engraftment and function of infused Th17 cells treated with distinct inhibitors were determined. Six days post-transfer, donor tumor-specific (Vβ14⁺) cells were detected in higher frequencies in the blood and spleen (FIG. 4F-H) when Th17 cells were primed in vitro with combination of Indo and CAL-101. Moreover these cells produced more effector cytokines, such as IFN-γ, IL-2, IL-17, upon PMA/Ionomycin activation ex vivo 64 days post-transfer (FIG. 4I). Taken together, these data indicate that expansion in vitro of ICOS-stimulated Th17 cells with Wnt/β-catenin and PI3K/p110δ pathway inhibitors augments their engraftment and antitumor activity in vivo.

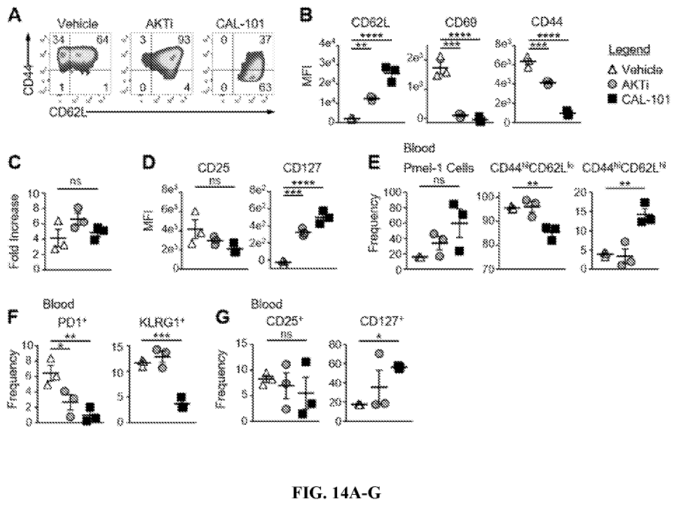

The engraftment and cytokine profile of donor inhibitor-treated Th17 cells was then assayed in vivo. Six days post-transfer, donor cells were detected in higher frequencies and absolute numbers in the spleen (FIGS. 13A-B) when primed in vitro with the Indo plus CAL-101. Moreover, these cells (and Indo alone treated Th17 cells) produced far more IFN-γ, IL-2 and IL-17A upon ex vivo re-activation 64 days post-transfer (FIG. 13C). These data indicate that treatment of ICOS-stimulated Th17 cells with p110δ plus β-catenin inhibitors paradoxically augments their function, persistence and antitumor activity in vivo.

Treatment outcome by Th17 therapy does not require host NK or CD8 T cells.

Two weeks post ACT, a marked but not significant increase in host NK cells in the tumor of mice treated with dual-inhibited Th17 cells was observed (FIG. 13D). Thus, it was asked whether host NK or CD8⁺ T cells play a role in this therapy with dual-inhibited Th17 cells. Interestingly, it was found that host NK and CD8 T cells may not contribute to treatment outcome, as antibodies depleting them for the entire experiment (˜2 months) did not compromise ACT therapy (FIGS. 13G-H) compared to mice treated with an IgG control (FIG. 13F). Mice depleted of host CD8 or NK cells experienced a profound antitumor response when infused with dual-inhibited Th17 cells. Curiously, not all mice (7 of 10) survived this therapy when treated with an IgG antibody control (FIG. 13F), suggesting that depletion of host NK or CD8 T cells provided additional space for the donor cells to thrive, a concept we and others have previously published regarding why lymphodepletion augments ACT (Paulos et al., 2007, Gattinoni et al., 2005). As expected, all mice succumb to disease if they were not treated with T cells (i.e. no treatment—FIG. 13E). These data may suggest that endogenous NK and CD8 T cells are not be key for the potent antitumor immunity mounted in mice infused with a dual drug-treated Th17 cells.

As mice infused with naïve TRP-1 transgenic CD4+ T cells have been reported to lyse tyrosinase positive melanoma in a MHC II restricted manner (Xie et al., 2010, Quezada et al., 2010), it was suspected that the dual-drug inhibited TRP-1 Th17 cells may directly regress tumors in vivo and thus depleting them would impair treatment outcome. To address this idea, mice were challenged a second time with B16F10 melanoma who survived long-term (˜50 days) from ACT therapy with dual-inhibited Th17 cells. These mice were either treated with a CD4 antibody to deplete donor cells (and host CD4) or were treated with an IgG antibody control. As shown in FIG. 131, only one of thirteen tumors grew in mice treated with an IgG antibody control. Importantly, the majority of these mice survived without tumor growth for an additional 50 days (˜100 days total). In contrast, tumors grew in most animals (9 of 13 mice) treated with a CD4 antibody, suggesting that donor T cells were responsible for the durable antitumor responses (FIG. 13J). Tumors grew rapidly in previously untreated “naïve” mice; regardless if they were CD4 depleted or not (FIGS. 13K-L). Collectively this reveals that donor Th17 cells pharmaceutically co-inhibited of p110δ and β-catenin can directly mediate profound antitumor activity against melanoma. However, the mechanisms underlying how co-inhibition of p110δ and β-catenin potentiates Th17 cells to regress tumor and persist long-term remains unknown.

Th17 Cells Express CD62L and Nominal FoxP3 when Treated with Inhibitors:

To uncover why inhibition of ICOS-induced pathways during in vitro Th17 cells expansion augments their antitumor efficacy in vivo, it was sought to determine how these drugs regulate their memory phenotype. Interestingly, it was found that CAL-101 treatment (day 8) supported the generation of central memory Th17 cells (CD44^{high}CD62L^{high}) (FIG. 5A, F). Moreover, Ly294002 (total PI3K inhibitor) also supported generation of T_{CM}17 cells (FIG. 5B, F). However, only specific inhibition of p110δ with CAL-101 dramatically increased CD62L expression and promoted a population of Th17 lymphocytes with a central memory profile. In particular, CAL-101 treated Th17 cells expressed elevated CCR7 and CD28 compared to control Th17 cells (FIG. 5C). They also expressed less CXCR3 and CCR6, which are markers for effector memory cells (FIG. 5C). In contrast to CAL-101, expansion of Th17 cells in vitro with Indo did not impact their memory phenotype (FIG. 5A-C). CAL-101 treatment appeared to dominate the profile of ICOS-activated Th17 cells, as those treated with both Indo and CAL-101 possessed a central memory phenotype (FIG. 5A-C, F).

Given that inhibition of PI3K pathway supports the generation of regulatory T cells (10,13), it was surmised that CAL-101 treatment would sustain FoxP3 expression in Th17 cells stimulated with ICOS. Conversely, it was posited that Indo would suppress FoxP3 expression, as previously reported (30). Surprisingly, CAL-101 treatment dramatically decreased FoxP3 and CD25 expression on ICOS-activated Th17 cells cultured 8 days, as well as on Th17 cells upon secondary stimulation in vitro (FIG. 5D, G). As expected, those cells expressed more FoxP3 and CD25 when treated with Ly294002 (FIG. 5E, G). Interestingly, concomitant Indo treatment suppressed FoxP3-induction caused by Ly294002 (FIG. 5E, G). Addition of Indo to CAL-101 therapy further decreased FoxP3 expression on resting as well as re-stimulated Th17 cells primed with ICOS (FIG. 5D, G).

Collectively, it was found that the ratio of central memory cells to suppressive FoxP3⁺ cells was significantly enhanced when ICOS-activated Th17 cells where expanded in vitro with Indo plus CAL-101 (FIG. 5H). These unique properties of enhanced central memory and reduced regulatory attributes in Th17 cells by these two drugs could offer an explanation for their robust capacity to eradicate tumors in vivo.

Tcf-7, Lef-1 and β-Catenin Rebound in Th17 Cells Treated with Indo:

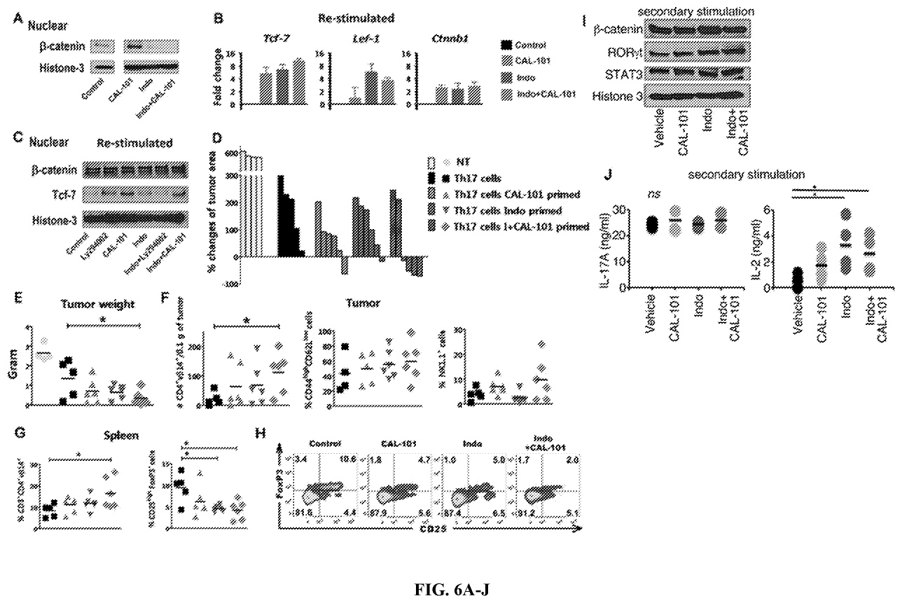

In agreement with Muranski et al., it was found that Th17 cells express β-catenin. It was also found that ICOS signaling induced β-catenin to even greater extent than CD28 co-stimulation (FIG. 1H). Given that CAL-101 treatment supports ICOS-activated Th17 lymphocytes with a central memory profile, it was hypothesized that they would express more β-catenin. Indeed, it was found elevated nuclear β-catenin expression upon CAL-101 treatment and as expected Indo abolish β-catenin translocation to the nucleus in those cells (FIG. 6A). Even though CAL-101 treated Th17 cell have higher β-catenin expression, which armed them with sternness, they were not as effective in vivo at killing tumors as the cells treated with both drugs. This in vivo data was particularly interesting given that Th17 cells treated with both compounds did not express β-catenin before transfer (β-catenin is directly associated with better treatment outcome). As reported elsewhere (26,27), Indo suppressed nuclear β-catenin translocation (FIG. 6A). However, β-catenin ablation by Indo was temporary and reversible, as the relative expression of Wnt/β-catenin downstream target genes was elevated upon secondary stimulation in vitro (refer as “re-stimulated” in the figure) in Th17 cells initially treated with drugs. A 12-fold increase of Tcf7, a 4-fold increase of Lef-1 and a 2-fold increase of Ctnnb1 gene expression was observed, when cells were treated with Indo and CAL-101 compared to control Th17 cells (FIG. 6B). The result was confirmed by Western blot analysis of nuclear protein in Th17 cells treated with inhibitors, as indicated. It was found that after antigen specific re-stimulation in vitro, Th17 cells initially expanded with CAL-101 and/or Indo re-expressed β-catenin to the level observed in un-treated ICOS stimulated Th17 cells (FIG. 6C). In agreement with the gene expression data, Th17 cells treated with CAL-101 or CAL-101 plus Indo expressed even higher levels of Tcf7 compared to control Th17 cells (FIG. 6C). RORγt and STAT3 exoression was also increased (FIG. 61).

As p110δ-induced Akt, β-catenin, and RORγt rebounded in Th17 cells (primed with CAL-101 and/or Indo) upon peptide re-stimulation, it was suspected they would regain their ability to secrete IL-17A. It was also expected that the cells would secrete IL-2: a cytokine produced by central memory T cells (Gattinoni et al., 2005(b)). Indeed, IL-17A production by these cells was restored (FIG. 13J). Dual inhibited Th17 cells secreted as much IL-17A as control Th17 cells. Remarkably, Th17 cells treated with Indo or with Indo plus CAL-101 also secreted significantly more IL-2 than untreated Th17 cells or CAL-101-treated Th17 cells (FIG. 13J). Our results provide evidence that β-catenin and PI3k/Akt signaling pathways rebound when infused into mice, licensing them to secrete IL-2 and persist.

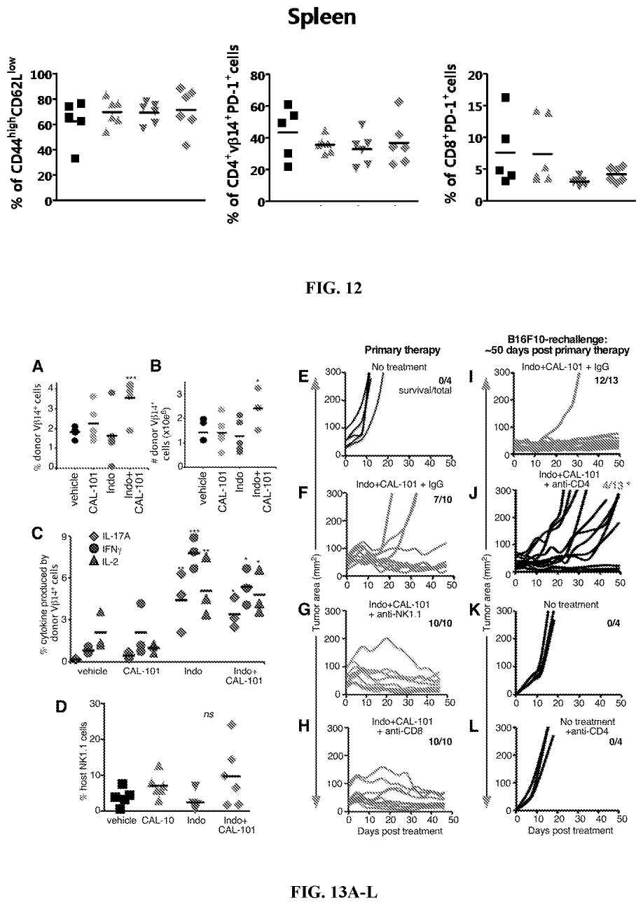

Noteworthy, the Ly294002 treatment could not replace the CAL-101 effect on Tcf7 overexpression. In support of the concept that ICOS-induced signaling pathways rebounded, it was also found restoration of cytokine production by ICOS activated Th17 cells after secondary stimulation in vitro (FIG. 12). Regardless of the method used for re-stimulation (splenocytes pulsed with trp-1 peptide, PMA/Ionomycin or B16F10 cancer cells) Th17 cells primed with inhibitors produced the same amount of IL-17A, IFN-γ, IL-2 as non-primed Th17 cells (FIG. 12). Th17 cells treated with both drugs were multi-functionality after re-stimulation, which was also observed the in vivo work (FIG. 4I).

It was hypothesized that PI3K and Wnt/β-catenin pathways rebounded in Th17 cells primed with both inhibitors, which ultimately augments treatment outcome. To address this concept in vivo, another persistence experiment was performed. Via a waterfall plot, it was found that ICOS activated Th17 expanded in vitro with Indo plus CAL-101 regressed tumors more efficiently than un-treated counterparts (FIG. 6D-E). Moreover, a higher frequency of those cells was found in the tumor and spleen (FIG. 6F-G) compared to control Th17 cells two weeks post-transfer. Interestingly, the β-catenin and phosphoAkt were re-expressed in vivo in donor Th17 cells expanded in vitro with drugs. Moreover, these cells also acquired effector memory phenotype in tumor and spleen (FIG. 6F & 12) upon ACT, which is necessary for them to confer potent antitumor immunity (31). Nonetheless, donor Vβ14⁺ Th17 cells primed in vitro with both inhibitors did not acquire an exhaustion profile in vivo, as Th17 cells treated with both drugs expressed less PD-1 in vivo. Interestingly, PD-1 expression on host CD8⁺ T cells was less in this treatment group (FIG. 12). The data also indicate that this therapy recruited NK cells but not CD8⁺ T cells or macrophages into the tumor site (FIG. 6F). Importantly, donor Th17 cells primed with Indo plus CAL-101 retained a less regulatory phenotype in vivo, as they expressed less FoxP3 and CD25 two weeks after transfer into mice (FIG. 6G-H).

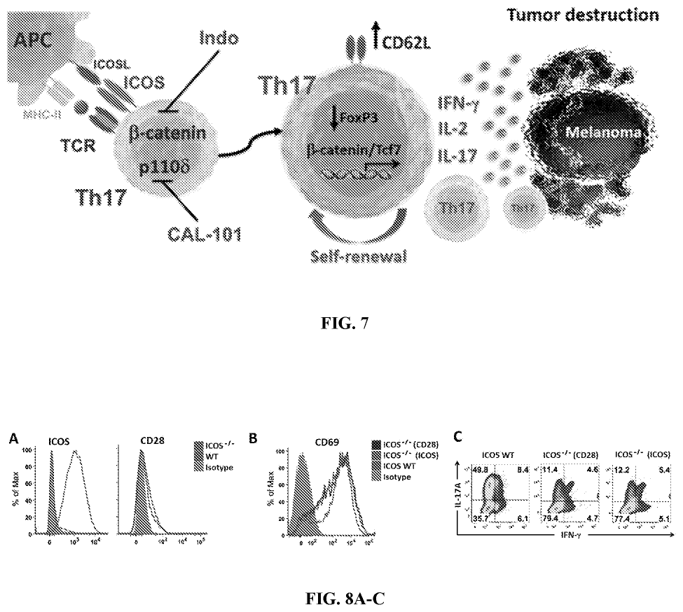

As depicted visually in FIG. 7, collectively, the data reveal that the treatment of ICOS stimulated Th17 cells with CAL-101 and Indo generates lymphocytes with an augmented memory phenotype (enhanced CD62L) and reduced regulatory properties (less FoxP3 and PD1). Moreover, these Th17 cells overexpressed genes and proteins in the Wnt/β-catenin pathway (β-catenin, Tcf7), in turn bolstering their multi-functionality and self-renewal, which ultimately enhanced their engraftment, persistence and potent tumor destruction when infused into mice with established melanoma (FIG. 7).

Herein, it was found that ICOS-stimulated Th17 cells persist long-term in vivo (>200 days), self-renew and mediate rapid recall responses against tumor re-challenge. ICOS stimulation induced two important survival pathways in Th17 cells: 1) PI3K/p110δ/Akt signaling pathway and 2) β-catenin in the Wnt signaling pathway. Interestingly, both pathways were reduced in Th17 cells genetically deficient in ICOS, underscoring its possible role in regulating these pathways in Th17 cells.

### Example 2—CAL-101 and AKTi Modified T Cells

Pharmaceutical Inhibition of p110δ in Pmel-1 CD8′ T Cells Increases Both CD62L and CD127 Expression without Hindering Expansion.

To test if PI3K6 inhibition enhanced the antitumor capacity of T cells similarly to direct AKT inhibition, CD8⁺ T cells from pmel-1 transgenic mice (CD8⁺ T cells with a transgenic TCR specific for the melanoma/melanocyte antigen gp100) were activated with their cognate antigen and treated with CAL-101 throughout culture. As controls, T cells were expanded without drug (vehicle) or with AKT inhibitor VIII (AKT 1/2 hereafter denoted as AKTi), a published method to enhance T cell memory and antitumor efficacy (Crompton et al., 2015). As expected, drug treatment induced marked differences in memory. While 65% of vehicle treated CD8⁺ T cells expressed CD62L, nearly all cells treated with AKTi or with CAL-101 expressed CD62L (FIG. 14A). However, CAL-101 exerted a stronger impact on the cells than direct AKT inhibition. The MFI of CD62L on CAL-101 treated T cells was higher than on those treated with AKTi. While both drugs decreased the activation marker CD69, only CAL-101 reduced CD44 expression (FIG. 14B). While this CD44¹⁰CD62L^{hi} phenotype mirrored the phenotype of a naive T cells, CAL-101 treated cells expanded similarly to both vehicle and AKTi treated cells. These data suggest that despite PI3K6 inhibition, T cell receptor and downstream proliferative signaling were intact in pmel-1 CD8⁺ T cells in vitro (FIG. 14C).

CAL-101 Treated CD8⁺ T Cells Robustly Engraft In Vivo and Maintain CD62L and CD127 Expression.

While AKTi and CAL-101 slightly reduced CD25 (IL-2 receptor alpha) on pmel-1 CD8⁺ T cells, these small molecules substantially elevated CD127, the alpha receptor for IL-7 (FIG. 14D). Since IL-7 signaling is important for naïve and central memory T cell homeostasis (Schluns et al., 2000), and supports donor T cells in the host (Johnson et al., 2015), experiments were performed to test whether memory T cells generated from either CAL-101 or AKTi treatment would show superior engraftment and subsequent antitumor immunity compared to vehicle. Interestingly, CAL-101 pmel-1 CD8⁺ T cells engrafted with increased frequency in the blood compared to AKTi pmel-1 CD8⁺ T cells (FIG. 14E). Additionally, while both AKTi and CAL-101 increased CD62L expression in vitro (FIG. 14B), only CAL-101 treated pmel-1 retained significant levels of CD44^{hi} CD62L^{hi} with fewer CD44^{hi}CD62L^{lo} circulating cells (FIG. 14E). Although both AKTi and CAL-101 T cells expressed less PD1 than vehicle in vivo, only CAL-101 T cells maintained reduced frequencies of the exhaustion marker KLRG1 (FIG. 14F). Infused CD8⁺ T cells also retained far more CD127 on their cell surface in vivo when primed ex vivo with CAL-101 compared to untreated or Akti treated pmel-1 CD8⁺ T cells (FIG. 14G). CAL-101 donor T cells were also detected at higher levels within the spleen and draining (inguinal) lymph nodes of tumor-bearing mice (not shown). Thus, it is shown that CAL-101 treated CD8⁺ T cells robustly engraft in vivo, maintain CD62L and CD127 expression and express lower levels of exhaustion markers.

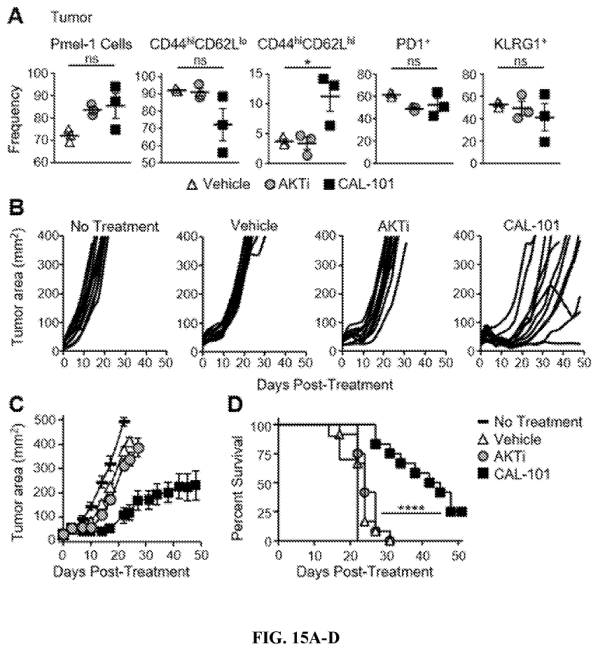

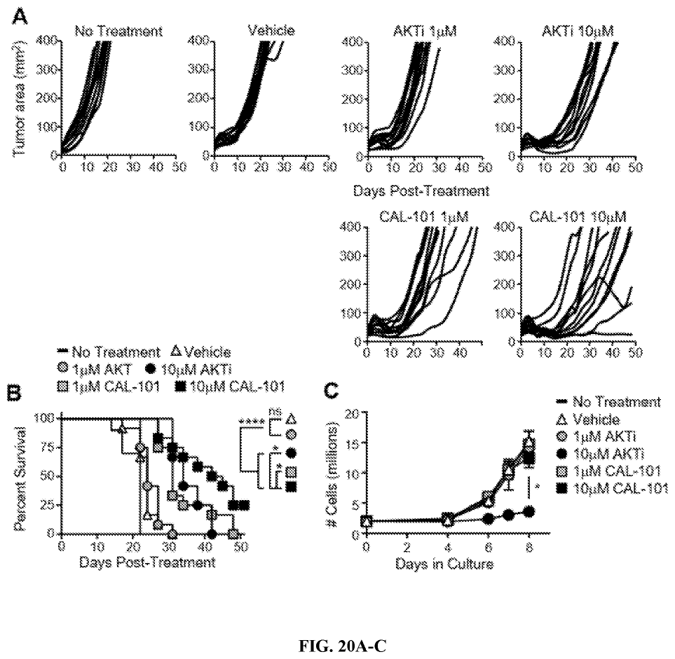

CAL-101 T cells impair tumor growth and prolong survival. We suspected that the less differentiated memory phenotype of pmel-1 T cells fostered by CAL-101 treatment would translate to their improved ability to infiltrate and regress tumor once infused into melanoma-bearing mice. As expected, both CAL-101 and AKTi treated T cells were detected at higher levels in the tumor compared to control (FIG. 15A). More CAL-101 donor cells expressed a central memory phenotype within the tumor, however, the percentage of PD1⁺ and KLRG1⁺ donor cells were similar between groups (FIG. 15A). Importantly, CAL-101 primed T cells were the most effective at slowing growth of melanoma in mice (FIG. 15B-C), extending the lifespan of the animals beyond the survival of the vehicle or AKTi groups (FIG. 15D). In fact, the tumor control exerted by AKTi treated T cells was only slightly improved over tumor control by vehicle treated T cells (FIG. 15C). Since a 10-fold higher drug dose was used in the CAL-101 cultures compared to the published amount of AKTi (10 μM vs 1 μM) it was hypothesized that the difference in antitumor activity could be due to higher AKT inhibition by the elevated concentration of CAL-101. To test this idea, pmel-1 CD8 T cells were treated with either 1 μM or 10 μM of AKTi or CAL-101. Increasing the amount of AKTi to 10 μM marginally improved the antitumor efficacy of the T cells similar to treatment with 1 μM CAL-101 (FIG. 20A). However, 10 μM CAL-101 treatment markedly improved tumor control and significantly improved survival compared to both 10 μM AKTi and 1 μM CAL-101 (FIG. 20A-B). Additionally, while neither 10 μM AKTi or 10 μM CAL-101 impaired the logarithmic expansion of mouse pmel-1 CD8⁺ T cells (not shown), we found that 10 μM AKTi dramatically inhibited growth of human T cells (FIG. 20C). Thus, as high dose AKTi did not profoundly improve antitumor T cell potency against large melanoma tumors but did compromise the overall yield of human T cell cultures, comparisons of 1 μM AKTi to 10 μM CAL-101 were used in the remaining studies.

CAL-101 Induces a Stronger Central Memory Phenotype than AKTi.

Next, it was examined whether inhibiting P131δ would augment the fitness and memory properties of human T cells. To do this, human CD3⁺ T cells were expanded with CD3/CD28 beads under CAL-101 or AKTi and compared to a vehicle control. All three T cell groups expressed similar CD45RO levels, a marker of T cell maturation. Both AKTi and CAL-101 treated T cells had slightly increased CD62L over vehicle, though only CAL-101 primed T cells had significantly higher CCR7 (FIG. 16A-B). PD1 was nominally expressed on all groups (not shown), but both drug treatments prevented the up-regulation of co-inhibitory receptor TIM3 post-activation compared to untreated T cells. Interestingly, CAL-101 significantly reduced TIM3 (FIG. 16C). P131δ and AKT blockade did not impact the expression of CD25, but only CAL-101 greatly increased CD127 on human T cells (FIG. 16C-D). Collectively, these data reveal that CAL-101 supports the generation of human T cells with a central memory phenotype with reduced markers of inhibition compared to treatment with AKTi.

PI3K8 Blockade Augments the Antitumor Activity of Human CAR-T Cells.

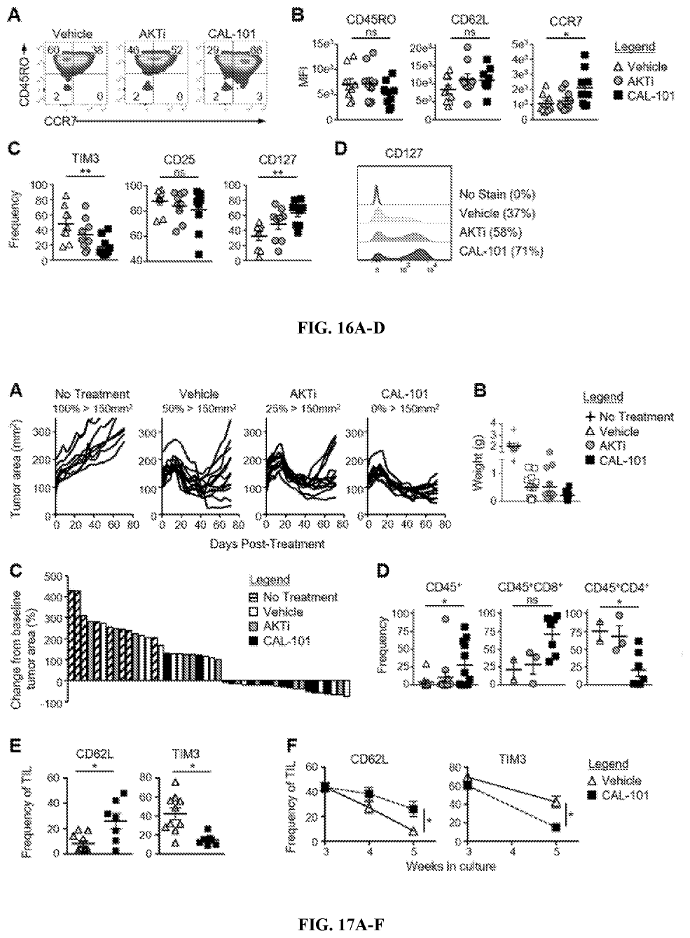

It was hypothesized that human tumor-reactive T cells treated with CAL-101 in vitro would control the growth of human tumors in NSG mice better than donor vehicle or AKTi T cells. To address this idea, human T cells were transduced with a lentiviral vector containing a chimeric antigen receptor (CAR) that recognizes mesothelin plus 4-1BB and CD3 signaling domains (Carpenito et al., 2009). These CAR T cells were expanded for seven days with CD3/CD28 beads and IL-2 in the presence or absence of CAL-101 or AKTi before transfer into mice bearing subcutaneous M108 mesothelioma tumor. While all treatment groups initially reduced tumor burden in the mice, CAL-101 treated CAR+ T cells exerted longer tumor control compared to the vehicle or AKTi treated groups (FIG. 17A). Conversely, half of tumors in vehicle T cell treated mice, and a quarter of AKTi T cell treated mice relapsed above 150 mm² (FIG. 17A). The superior antitumor immunity from CAL-101 primed T cells was further evidenced by the majority of tumors in CAL-101 mice having the smallest mean tumor weight (FIG. 17B) and remaining below baseline measurement at the end of study (FIG. 17C). Additionally, CAL-101 T cells persisted at significant levels in circulation 55 days after transfer in most treated mice (FIG. 17D). This finding with CAL-101 was in stark contrast to vehicle and AKTi T cell treated mice, which showed low levels of persisting cells in the mice (FIG. 17D). Thus, priming T cells with CAL-101 improves engraftment, persistence and tumor destruction by human CAR T cells in solid tumors.

Since P131δ inhibition improved the antitumor efficacy of healthy donor derived CAR T cells, it was hypothesized that CAL-101 treatment would also improve the memory phenotype of tumor infiltrating lymphocytes (TIL) from patients with lung carcinoma. To address this concept, individual TIL cultures were expanded under IL-2 for three weeks then split into CAL-101 or vehicle treatment groups for another two weeks. It was found that CAL-101 supported the generation of TIL with higher CD62L but low TIM3 expression than vehicle after 5 weeks of expansion (FIG. 17E). This phenotype appeared to be due to preservation of a less differentiated memory phenotype as vehicle TIL lost expression of CD62L faster than CAL-101 TIL (FIG. 17F). Additionally, while TIM3 expression on both groups diminished during ex vivo expansion, CAL-101 treatment mediated a rapid loss of TIM3 on TILs (FIG. 17F). Collectively, these data reveals that P131δ blockade augments the antitumor activity of human CAR-T cells and fosters the generation of TILs with a less exhausted and preserved memory profile.

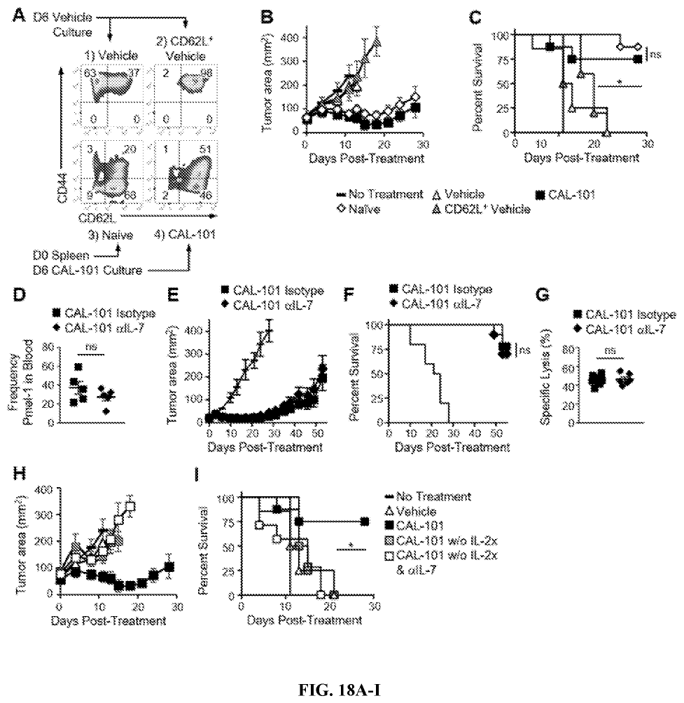

Antitumor potency induced by PI3Kδ inhibition is not due to CD62L. CD62L expression on T cells correlates with improved antitumor immunity in pre-clinical ACT tumor models (Gattinoni et al., 2005(b); Berger et al., 2008; Hinrichs et al., 2011; Klebanoff et al., 2016; Sommermeyer et al., 2016). Moreover, enriching central memory T cells from peripheral blood and redirecting them with a CD19 specific CAR has shown efficacy in a clinical trial (Wang et al., 2016). The studies presented herein corroborated these reports by showing a correlation between retained CD62L expression in vivo by CAL-101 treated donor cells and prolonged tumor control (FIGS. 14 & 15). Consequently, it was hypothesized that CAL-101 induced CD62L on T cells was responsible for their enhanced antitumor potency. Thus, if this concept were true, it is suspected that simply sorting the CD62L⁺ T cells from vehicle pmel-1 should mirror the antitumor activity of CAL-101 primed T cells, which express high CD62L. To test this, the following pmel-1 T cell cohorts were administered to B16F10 mice: 1) bulk vehicle T cells (which were 37% CD62L⁺), 2) sorted CD62L⁺ T cells from vehicle treated T cells (98% CD62L⁺), 3) naïve pmel-1 T cells sorted directly from the spleen (majority CD44⁻CD62L⁺), and 4) CAL-101 treated T cells (which were 97% CD62L⁺, see FIG. 18A).

In contrast to the hypothesis, it was found that sorting CD62L⁺ pmel-1 cells from vehicle cultures did not improve treatment outcome over therapy with bulk vehicle. Rather, CAL-101 mediated prolonged antitumor control (FIG. 18B) and better survival (FIG. 18C) in mice. Interestingly, naïve sorted T cells closely matched the CAL-101 treated T cells in antitumor potency. It is important to note that despite similar antitumor efficacy, the benefit of CAL-101 expanded T cells over enriched naïve T cells is the potential for propagating signification more cells (i.e. higher cell yield) as vast numbers of cellular product are paramount for successful ACT therapy (Gattinoni et al., 2005(b); Bowers et al., 2017). Nonetheless, these results imply that PI3Kδ blockade during in vitro proliferation might preserve naïve T cell qualities, which would otherwise be corrupted by the expansion process. This finding also corroborates the data from TIL indicating a slower decline in CD62L in CAL-101 treated TIL. Thus, while T cells capable of long-lived memory responses against tumor express CD62L, enriching cells after ex vivo expansion that expresses this molecule is not sufficient to drive a successful antitumor response.

CAL-101 primed T cells regress tumor independent of IL-7 signaling.

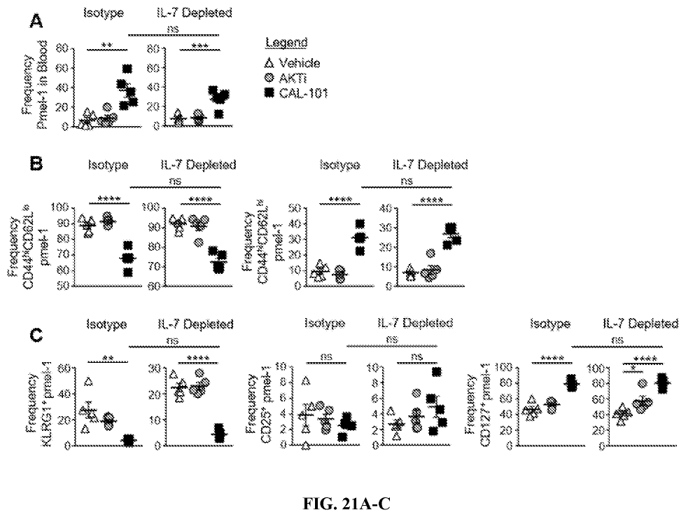

Next it was tested whether the preserved CD127 on CAL-101 treated T cells was responsible for the robust antitumor properties in vivo. PI3Kδ blockade induced CD127 on murine and human T cells in vitro. Moreover, CD127 was sustained on CAL-101 treated T cells after transfer (FIGS. 14E and 15A). It was hypothesized that CAL-101 T cells thrived in vivo due to their enhanced responsiveness to IL-7. To test the dependence of CAL-101 antitumor responses on IL-7 signaling, we depleted IL-7 in the pmel-1 B16F10 model for the first two weeks after transfer as published (Johnson et al., 2015). It was suspected that depleting IL-7 would decrease engraftment of the donor cells and impair their control of tumor growth. In contrast, while CAL-101 primed T cells engrafted at significantly higher numbers than vehicle or AKTi treated T cells in both isotype and IL-7 depleted mice (FIG. 21A), there was no significant difference between the CAL-101 treatment groups in frequency or memory phenotype (FIGS. 18D & 21B-C). CAL-101 primed T cells also exerted their prolonged antitumor immunity whether IL-7 had been depleted or not (FIG. 18E) resulting in no differences in survival between the CAL-101 T cell groups (FIG. 18F). Furthermore, while IL-7 is important for maintaining naïve and central memory T cell populations, no reduction was found in memory capacity of donor CAL-101 T cells in either isotype or IL-7 depleted animals, as both groups were equally capable of lysing tumor in mice (FIG. 18E-F) and ablating hgp100 antigen bearing splenocytes in our very sensitive in vivo cytotoxicity assay (FIG. 18G).

As IL-2 complex was administered to our melanoma tumor-bearing mice to support the infused CAL-101 T cells, it was suspected that this cytokine was important for the engraftment of these infused cells and could compensate for IL-7. IL-2 complex has been reported to support the engraftment and proliferation of CD8⁺ T cells in ACT murine models (Boyman et al., 2006). Thus, it was hypothesized that removal of IL-2 complex from the treatment protocol would reveal the importance of IL-7 signaling in the antitumor efficacy mediated by CAL-101 T cells. As expected, tumors grew more rapidly in mice that received CAL-101 T cells without IL-2 complex, compared to those which received both the pmel-1 CD8⁺ T cells and IL-2 complex (FIG. 18H). However, because none of the pmel-1 T cell treatments without IL-2 complex were therapeutic, it was not possible to truly assess if IL-7 depletion compromised the antitumor activity of CAL-101 pmel-1 T cells in the absence of exogenous IL-2 administration (FIG. 18H-I). Nonetheless, these experiments highlight the central importance of bolstering IL-2 signaling for effective antitumor function of adoptively transferred CD8⁺ T cells treated with CAL-101. Collectively, these findings lend further evidence that improved antitumor T cell efficacy from CAL-101 treatment is not likely due to their increased responsiveness to IL-7 signaling due to high CD127 expression nor was their improved potency due solely to their heighted expression of CD62L.

CAL-101 Induces Stem Memory Pathways in T Cells while AKTi does not.

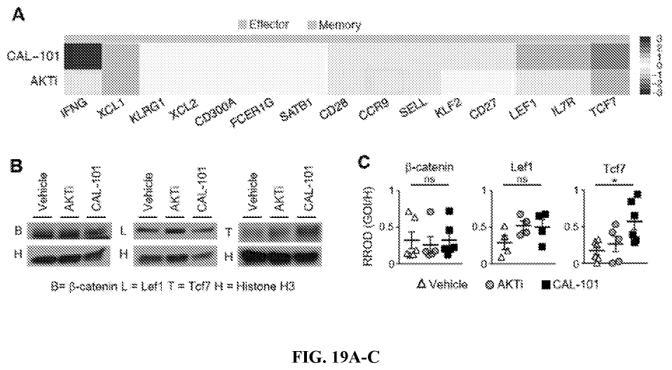

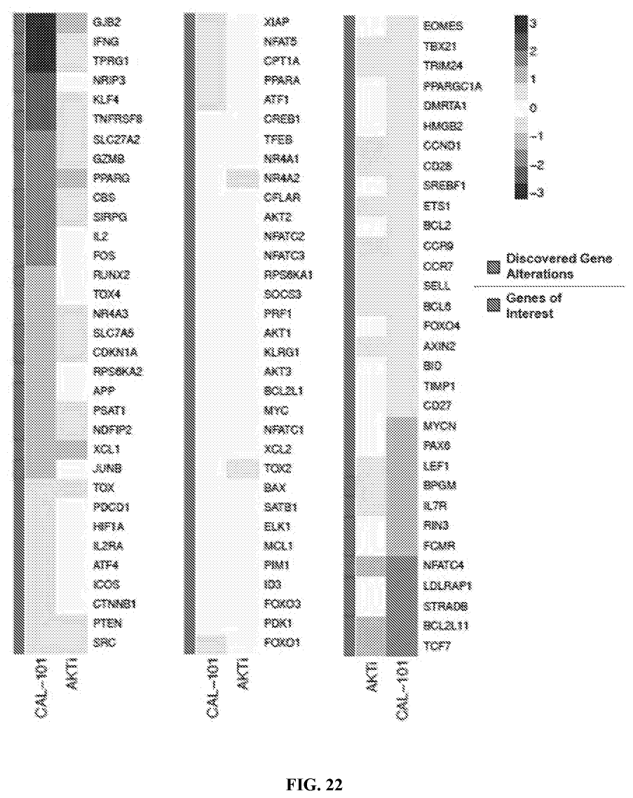

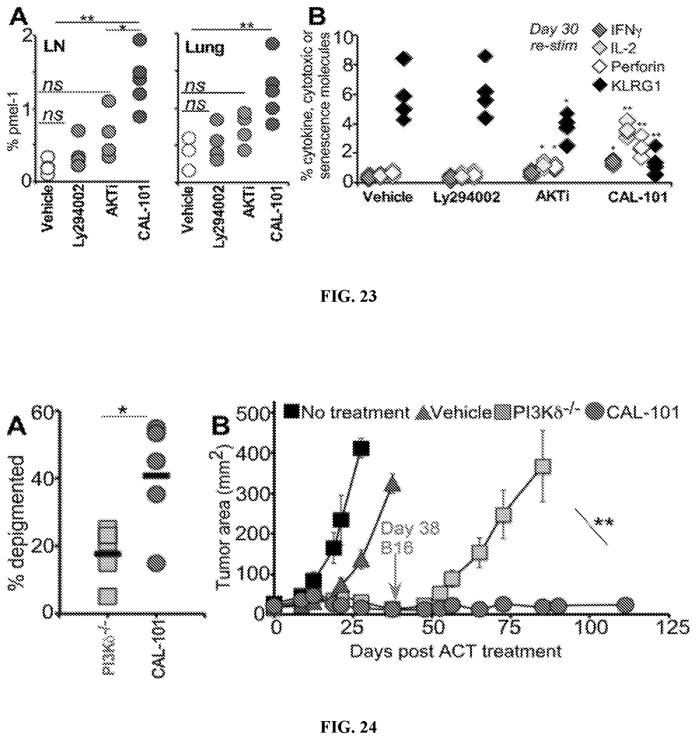

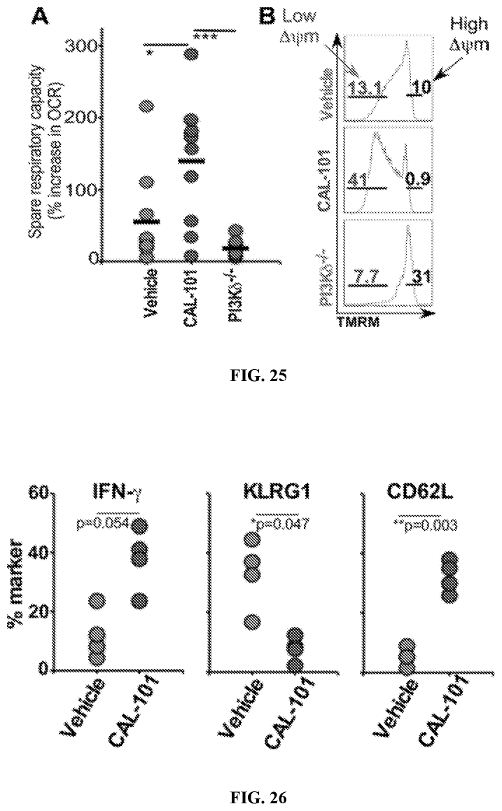

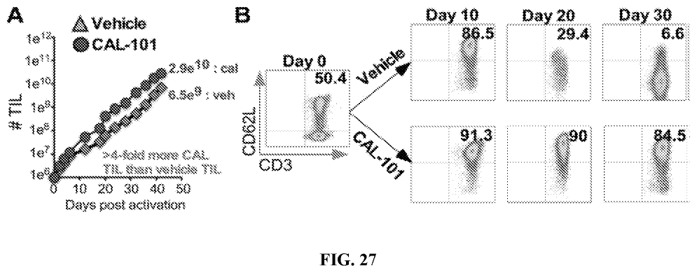

To define how CAL-101 instills infused CD8⁺ T cells with enhanced antitumor activity in vivo, RNA sequencing was used to uncover the factors potentially responsible for the efficacy of this ACT therapy. Differential expression of RNA transcripts of interest associated with memory and effector phenotypes were surveyed, as well as other pathways influenced by drugs that block PI3K or AKT, including signaling intermediates, metabolic, anti-apoptotic pathways and cell cycle proteins. Similar to the protein data, PI3Kδ inhibition via CAL-101 promoted the up-regulation of multiple central memory markers on T cells, such as CD62L (Sell) and CCR7 compared to AKTi or vehicle-treated T cells. CAL-101 also uniquely induced high CD127 (IL7r) transcript and stem memory associated transcripts Lef1 and Tcf7, which were markedly increased compared to AKTi cells (FIG. 19A and FIG. 22A). A durable memory phenotype would equate to decreased expression of transcripts associated with differentiate T cell effector function. In most cases, as expected, both drug treatments down-regulated effector transcripts including Fos, JunB, Granzyme B (Gzmb) and IFN-γ (Ifng), but interestingly the effector transcription factors Tbx21, Eomes and Nfatc4 increased with CAL-101 treatment (FIG. 19A and FIG. 22A).

The high expression of stem memory genes Lef1 and Tcf7 in CAL-101 treated T cells, known to enhance their persistence and antitumor activity (Gattinoni et al., 2011; Bowers et al., 2017; Maichrzak et al., 2017), was found particularly intriguing, so the RNA-seq data was followed up by assaying the protein levels of nuclear Lef1, Tcf7 and their upstream molecule β-catenin in CAL-101 treated T cells versus AKTi and vehicle T cells. Similar to the transcript expression results, nuclear β-catenin was similar between groups and both drug treatments had slightly higher, though not statistically significant, Lef1. However, PI3Kδ blockade significantly increased nuclear Tcf7 over vehicle while AKTi did not (FIGS. 19B-C). These results confirm the findings with RNA-seq, and collectively suggest that while the phenotype of AKT inhibited T cells resembles that of potent central memory T cells, PI3Kδ blockade profoundly induced key stem memory transcripts and protein in human CAR T cells that might be fundamentally responsible for their longevity and potency in vivo.

In addition to memory transcripts that were hypothesized would be altered by CAL-101 treatment (designated as “Genes of Interest” see FIG. 22A), other transcripts were detected which were differentially expressed in CAL-101 primed T cells vs vehicle (designated as “Discovered Gene Alterations” see FIG. 22A). Many of these genes are associated with T cell fitness versus exhaustion including high anti-apoptotic and metabolism transcripts Bcl2, stradb (or ILPIP) and Ldlrap1 (FIG. 22A). Interestingly, ILPIP is an anti-apoptotic protein with energy-generating metabolism (Sanna et al., 2002). Of additional interest, KLF4 was dramatically down regulated in T cell treated with CAL-101. This finding is important, as this transcript has been previously reported to restrict memory CD8⁺ T cell responses to foreign antigen (Mamonkin et al., 2013) (FIG. 22A). Also, cell cycle proteins were found to be differentially regulated, including cyclin D1 (CCND1) which was upregulated uniquely with CAL-101 treatment, and p21 (CDKN1A) which was downregulated compared to control especially by CAL-101 treatment. Since p21 prevents cyclin protein mediated progression from G1 to S (Deng et al., 1995) and increased transcript of cyclin D1 was observed, suggesting that it is possible that the increased cell frequencies of CAL-101 T cells after infusion may be due in part to less inhibited cell division (FIG. 22A). Collectively based on this data, it is suspected that CAL-101 induced Tcf7 signaling while concomitantly down regulating KLF4 may augment memory and antitumor efficacy by preventing the cells from undergoing terminal differentiation. Thus, CAL-101 regulates distinct pathways (such as Tcf7, KLF4 & ILPIP) potentially critical for supporting a less differentiated memory phenotype in adoptively transferred T cells.

### Example 3—Materials and Methods

Mice and tumor lines: Six- to 10-week-old male TRP-1 TCR-transgenic mice, eight-week old female C57BL/6 mice, C57BL/6J (B6), pmel-1 TCR transgenic mice and NOD/scid/gamma chain knock out (NSG) mice were purchased from The Jackson Laboratory. Mice were housed in the Hollings Cancer Center (Charleston, S.C.) vivarium and maintained in compliance with the Medical University of South Carolina (MUSC; Charleston, S.C.) Institutional Animal Care and Use Committee (IACUC). NSG mice were housed under specific pathogen-free conditions in microisolator cages and given autoclaved food and acidified water. The experimental procedures here in we reapproved by the IACUC. The B16F10 tumor line was obtained as a gift from Dr. Nicholas Restifo at the National Cancer Institute, National Institutes of Health, Bethesda, Md. M108 xenograft tumors were obtained as a gift from the June lab at the University of Pennsylvania. M108 were cultured and engrafted as described previously (Carpenito et al., 2009).

Cell Preparation and Culture:

Splenocytes were harvested from Vβ14⁺ TRP-1 TCR-transgenic mice. Cells were cultured in RPMI-1640 medium with 10% FBS, 2 mmol/L L-glutamine, 1% Na pyruvate, 1% nonessential amino acids, 0.1% HEPES, 1% penicillin, 1% streptomycin, and 0.1% 2-β-mercaptoethanol (all from Sigma-Aldrich). TRP-1 splenocytes were activated with beads coated with anti-mouse CD3 (clone 145.2C11, BioLegend) and with CD28 agonist (Clone 37.51, BioLegend) or ICOS agonist (Clone 398.4A, BioLegend). On day 0 cells were polarized toward Th17 phenotype using 10 ng/mL rhIL-1β, 100 ng/mL rmIL-6, 100 ng/mL rmIL-21, 30 ng/mL rmTGFβ, 10 μg/mL anti-mouse IL-4, 10 μg/mL anti-mouse IFNγ. T cells were split every day starting from day 3 and supplemented with 100 IU/mL rhIL-2 and 10 ng/ml rmIL-23 (R&D). Distinct subsets were harvested on the days indicated and used for gene expression analysis (Real-time PCR), protein expression analysis (Western blot), flow cytometry or in vivo studies. De-identified human PBMCs and tumor samples were collected under approval of the MUSC Internal Review Board. Human T cells were engineered via approval from the Institutional Biosafety Committee.

T Cell Cultures.

Pmel-1 CD8⁺ T cells were prepared from whole splenocytes activated using 1 μM hgp100 peptide+100 IU rhIL-2/mL. Starting 3 hours after initial activation, cells were treated with either DMSO vehicle, AKT inhibitor VIII (AKTi) (Calbiochem), or CAL-101 (Selleckchem) at indicated doses. Cells were supplemented with culture media containing 100 IU rhIL-2/mL and vehicle or drug when expanded. Human Normal Donor Peripheral T cells: Polyclonal CD3⁺ T cells were prepared via negative bead selection (Dynal) from peripheral blood lymphocytes and activated using CD3/CD28 beads (Gibco) with 100 IU rhIL-2/mL DMSO vehicle, 1 μM or 10 μM AKTi or CAL-101 throughout culture as indicated. On day 2 of culture, T cells were engineered to be mesothelin specific via lentiviral transduction with an anti-mesothelin chimeric antigen receptor (CAR) which contained a single-chain variable fragment (scFv) fusion protein specific for mesothelin and linked to the T cell receptor ζ (TCRζ) signaling domain and 4-1BB as described previously (Carpenito et al., 2009) (gifts from the June lab).

Tumor Infiltrating Lymphocytes.

Tumor infiltrating lymphocytes (TILs) were derived from non-small cell lung cancer tumor samples from two patient donors provided by Dr. John Wrangle and Dr. Mark Rubinstein. The tumors were rinsed in CM and cut into 1-3 mm pieces, which were individually transferred to wells of a 24-well plate containing 2 ml of TIL media (CM with a final concentration of 6,000 IU/mL recombinant IL-2). Each well was considered an individual TIL product for analysis. After 5-7 days, 1 ml of media was removed and replaced with 1 ml of fresh TIL media. TIL were monitored and either given fresh TIL media or split when confluent every 2-3 days for up to 5 weeks. On week 3, half of split wells were given CAL-101 (10 μM), while the original wells were treated with vehicle for the duration of the experiment.

Expansion of Th17 cells with Wnt/β-catenin and PI3K inhibitors in vitro: TRP-1 splenocytes were activated with irradiated (10Gy) C57BL/6 splenocytes pulsed with TRP-1 peptide (1 μM) added at a 5:1 ratio in 24-well plates (1 mL media containing 1.5×10⁶ cells/well). TRP-1 splenocytes were co-stimulated with soluble ICOS agonist (Clone 398.4A, BioLegend) and polarized toward Th17 phenotype as describe above. For in vitro expansion from day 0 of culture 10 μM Ly294002 (Cayman Chemical) or 10 μM CAL-101 (Selleckchem) and/or 60 μM indomethacin (Sigma-Aldrich) was added. Also dimethylosulfoxide 0.01% (Sigma-Aldrich) was added as a control. Th17 cultures were also supplemented with hrIL-2 (100 IU/mL) and rmIL-23 (long/ml; R&D). Medium, including cytokines and inhibitors was refreshed every 2-3 days.

Real-Time PCR:

Total RNA isolation was performed using the Trizol method (Life Technologies) according to the manufacturer's procedure. RNA quantity and purity was assessed using a NanoDrop ND-1000. 1 μg total RNA was transcribed with Transcriptor First Strand cDNA Synthesis Kit (Roche). qReal-time PCR was performed on LightCycler480 machine following manufacturer's protocols (Roche). Probes utilized include Rorc, IL-17, Cmaf IL-21, Cpt-1a, Tcf-7, Lef-1, Cnnb1. All probes are commercially available (ABI). Relative gene expression was determined by the comparative CT method (45). β-actin was used as housekeeping gene.

Western Blot Analysis:

Nuclear and cytoplasmic protein were isolated by lysis using Nuclear and Cytoplasmic Extraction Reagent with Protease&Phosphatase Inhibitor Cocktail (ThermoScientific). Protein concentration was quantified using BSA Protein Assay (ThermoScientific) according to the manufacturer's instructions. 10 to 30 μg of total protein was separated on a Mini-PROTEAN TGX, Any kD™ gel, followed by transfer onto PVDF membrane (Bio-Rad). The membranes were blocked with 5% BSA in TBS buffer containing 0.5% Tween20. The membranes were then incubated overnight at 4° C. with the primary antibodies to β-catenin (BD Bioscience), p110δ, Tcf7 (Abcam), RORγt (Affymetrix) phospho-Akt, total-Akt, STAT-3, Histone-3, Lef1 (abcam), and GAPDH (Cell Signaling) at the concentration recommended by manufactures. Subsequently, membranes were washed and incubated for 1 h at room temperature with (HRP)-conjugated goat antibodies to mouse or rabbit IgG (Cell Signaling). For detection of protein, chemiluminescence method was performed using Western ECL Blotting Substrate (Bio-Rad) followed by X-ray film-based imaging method (ThermoScientific). Quantification of optical density performed using Fiji analysis software (NIH).

Flow Cytometry:

Surface staining of Th17 cells was performed with anti-CD4-APCCy7, anti-Vβ14-FITC (BD Biosciences) anti-CD62L-APC, anti-CD44-PerCPCy5.5, anti-CCR6-PE, anti-CD69-PECy7, anti-CCR7-PE, anti-CD27-PE, anti-CD28-PerCPCy5.5, anti-ICOS-PE and anti-CD25-PECy7/FITC (BioLegend) and viability stain (Invitrogen) on day 8. For all intracellular staining of cytokines (anti-IL-17 PerCPCy5.5, anti-IFN-γ-v450, anti-IL-2-FITC, anti-IL-21-APC, anti-IL-22-PE, anti-TNF-α-FITC; BioLegend) and transcription factors (anti-RORγt-APC, anti-cMaf-PerCPCy5.5, anti-STAT-3-PE, anti-FoxP3-FITC/PE, anti-T-bet-v450; eBiosciences), cells were stimulated for 4-5 h with 50 ng/mL PMA and 750 ng/mL ionomycin (Sigma-Aldrich). Additional antibodies used include: CD127-PECy7 clone A019D5, CD25-APCCy7 clone BC96, CD4-APCCy7 OKT4, CD45RO-APC clone UCHL1, CD62L-FITC clone DREG-56, CD8-PerCPCy5.5 clone SKI, TIM3-PE clone F38-2E2 (biolegend); CCR7-PECy7 clone CCR7, CD8-V450 clone RPA-T8, PD1-FITC clone M1H4, (BD Biosciences). Antibodies used for mouse cell analysis: CD127-PE/V450 clone A7R34, CD25-FITC clone 7D4, CD44-PerCPCy5.5/FITC clone IM7, CD69-PECy7 clone HI.2F3, CD8-PerCPCy5.5 clone 53-6.7, CD8-PECy7 clone YTS156.7.7, PD1-PerCPCy5.5 clone 29F.1A12 V□13-PE/APC clone MR12-3/MR12-4 (Biolegend), CD4-APCCy7 clone RM4-5, CD62L-APC clone MEL-14, KLRG1-APC/V450 clone 2F1 (BD). After 1 h, Monensin (BioLegend) was added per manufacturer's instructions. After surface staining, intracellular staining for cytokines and transcription factors was performed using Fixation and Permeabilization buffers (BioLegend) and Foxp3 Staining Buffers (eBioscience), respectively. Flow cytometry acquisition was performed on a FACSVerse or Accuri (BD Bioscience). Data were analyzed with FlowJo software (TreeStar). For experiments with sorted cells, CD8⁺Vb13⁺ T cells were sorted from pmel-1 cultures using Dynabeads untouched mouse CD8⁺ T cell kit (Invitrogen) and a FACSAria cell sorting machine (BD Biosciences).

ELISA:

Cytokine levels were measured using the mouse IL-17A, INF-γ, IL-2, IL-9, IL-10, IL-21, IL-22, CCL-20, GM-CSF ELISA kits (R&D) following the manufacturer's protocol.

Adoptive Cell Transfer Tumor Treatment Experiments:

C57/B6 mice were inoculated subcutaneously with 3×10⁵ B16F10 melanoma cells. 10-12 days later 1×10⁶ TRP-1 CD4⁺ T cells programmed toward Th17 phenotype, co-stimulated with ICOS vs. CD28 or 0.75×10⁶ TRP-1 Th17 cells expanded in vitro for 8 days in the presence or absence of CAL-101 or Ly294002 and/or Indo, were transferred via tail vein injection. Recipient animals were sub-lethally irradiated (5Gy) prior to adoptive cell transfer. Tumors were measured using calipers, and the perpendicular diameters were recorded. Experiments were repeated twice, with similar results.

Adoptive Cell Therapy.

C57/B6 mice received 4.5×10⁵ B16F10 cells subcutaneously 5-8 days before ACT. One day before therapy, mice underwent nonmyeloablative 5 Gy total body irradiation. CD8⁺ T cells were in vitro activated with feeder cells and peptide 12 hours before transfer as described (Klebanoff et al., 2009) then infused via tail vein. Unless otherwise indicated, IL-2 complex was prepared at 1.5 μg rhIL-2 (NIH) and 7.5 μg anti-IL-2 antibody (clone JES6-1A12 BioXCell) per mouse and administered via intraperitoneal injections on days 0, 2, and 4 of treatment. Mice in the antibody neutralization experiment received 200 μg of either IL-7 neutralizing antibody (clone M25) or IgG2b isotype (clone MPC-11) (BioXCell) on days 0, 3, 5, 8, 12, and 17 of treatment via intraperitoneal injection as previously described (Johnson et al., 2015). NSG mice received 6×10⁶ M108 suspended in matrigel subcutaneously 51 days prior to adoptive therapy. On day of treatment, mice received 3.5-4×10⁵ CAR T cells via tail vein injection. In all experiments, mice were randomized to treatment groups and tumor burden was monitored in blinded fashion using perpendicular caliper measurements. Tumor burden was reported as tumor area (mm²).

Engraftment and Persistence:

A total of 1×10⁶ Th17-polarized TRP-1 cells co-stimulated with ICOS vs. CD28 or stimulated with ICOS and expended for 8 days in the presence or absence of CAL-101 and/or Indo, were transferred into 5Gy irradiated C57/B6 mice bearing 10 day established tumors. Blood, spleens, lungs, inguinal lymph nodes and tumors were collected at indicated days and homogenized. Cells were enumerated using trypan blue exclusion. Frequency of Vβ14⁺CD4⁺ cells was analyzed by flow cytometry. Surface staining of Th17 cells was performed with anti-CD4-APCCy7, anti-Vβ14-FITC, anti-CD8-APC (BD Biosciences) anti-NK1.1-PE, anti-CD44-PerCPCy5.5, anti-CD62L-APC, anti-CD25-PECy7, anti-PD-1-PerCPCy5.5 (BioLegend) and viability stain (Invitrogen). For cytokine secretion (anti-IL-17-PerCPCy5.5, anti-IFN-γ-v450, anti-IL-2-PE) cells were stained according to the manufacturer's protocol using Fix and Perm buffers (BioLegend). The experiment with Th17 cells treated in vitro with drugs was performed three times with similar results.

Tissue distribution assays.

Blood from treated mice was collected via cheek vein bleed then centrifuged to remove plasma and subjected to RBC lysis buffer (Biolegend), before being re-suspended in cell media for analysis. Spleens and draining (inguinal) lymph nodes were prepared via mechanical disruption followed by red blood cell lysis, and re-suspended for analysis. Tumors were sectioned, then mechanically disrupted and re-suspended in for analysis. Cell suspensions were blocked using FC block (Biolegend) at 1 μg/100 μL prior to probing with antibodies, then analyzed by flow cytometry.

RNA Sequencing and Analysis.

Library preparation: mRNA libraries were prepared in triplicate from each donor using the TruSeq RNA V2 kit(Illumina). Cleaved RNA fragments were copied into first strand cDNA then underwent second strand cDNA synthesis. End repair of cDNA fragments, single ‘A’ base addition and ligation to the adapter followed. The product was then purified and enriched with PCR to create the final cDNA library. Transcriptome sequencing: cDNA libraries were clonally clustered onto the sequencing flow cell using the c-BOT (Illumina) Cluster Generation Station and Hiseq Rapid Paired-End Cluster Kit v2 (Illumina). Clustered flow cells were sequenced on the Illumina HiSeq2500 Sequencing System using the Hiseq Rapid SBS Kit V2 (Illumina). Analysis: Differential gene expression analysis contrasting the factors representing different treatment protocols (CAL101 vs Vehicle; or AKTi vs Vehicle) was performed by running kallisto on the raw paired-end RNA sequencing data (in FASTQ format) to estimate transcript-level read counts based on Ensembl GRCh37 cDNA assembly for each sample (NCBI GEO database Accession Number: GSE101497). Estimated transcript-level counts were aggregated across official gene symbols using the tximport package with mapping provided by the biomaRt utility. To better isolate the effects of drug treatments from the effects that can be attributed to differences in donors, we used both factors in our modeling of the gene-level count levels and used limma voom package to fit our model following their published standard protocol (Law et al., 2014).

Statistics.

Kaplan-Meier survival curves were assessed for significance using a log rank test between treatment groups. A p-value of <0.05 was considered significant. Statistical comparisons between groups were performed via a student's t-test for 2 groups, or a one-way ANOVA followed by multiple comparisons of group means (3+ groups). A p-value of <0.05 was considered significant. All statistics reported as mean±SEM. Statistical analysis of differential gene expression was performed using Bonferroni-adjusted p-values to account for multiple hypothesis testing and a cut-off at 0.05 for the adjusted values to assign significance of the differential expression across conditions.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Muranski P, Boni A, Antony P A, Cassard L, Irvine K R, Kaiser A, et al. Tumor-specific Th17-polarized cells eradicate large established melanoma. Blood 2008; 112(2):362-73.
2. Martin-Orozco N, Muranski P, Chung Y, Yang X O, Yamazaki T, Lu S, et al. T helper 17 cells promote cytotoxic T cell activation in tumor immunity. Immunity 2009; 31(5):787-98.
3. Muranski P, Borman Z A, Kerkar S P, Klebanoff C A, Ji Y, Sanchez-Perez L, et al.

Th17 cells are long lived and retain a stem cell-like molecular signature. Immunity 2011; 35(6):972-85.
4. Park H, Li Z, Yang X O, Chang S H, Nurieva R, Wang Y H, et al. A distinct lineage of CD4 T cells regulates tissue inflammation by producing interleukin 17. Nature immunology 2005; 6(11):1133-41.
5. Tanaka S, Suto A, Iwamoto T, Kashiwakuma D, Kagami S, Suzuki K, et al. Sox5 and c-Maf cooperatively induce Th17 cell differentiation via RORgammat induction as downstream targets of Stat3. The Journal of experimental medicine 2014; 211(9): 1857-74.
6. Stritesky G L, Yeh N, Kaplan M H. IL-23 promotes maintenance but not commitment to the Th17 lineage. Journal of immunology (Baltimore, Md.: 1950) 2008; 181(9):5948-55.
7. Perry J M, He X C, Sugimura R, Grindley J C, Haug J S, Ding S, et al. Cooperation between both Wnt/{beta}-catenin and PTEN/PI3K/Akt signaling promotes primitive hematopoietic stem cell self-renewal and expansion. Genes & development 2011; 25(18):1928-42.
8. Kryczek I, Zhao E, Liu Y, Wang Y, Vatan L, Szeliga W, et al. Human TH17 cells are long-lived effector memory cells. Science translational medicine 2011; 3(104):104ra00.
9. Paulos C M, Carpenito C, Plesa G, Suhoski M M, Varela-Rohena A, Golovina T_{N}, et al. The inducible costimulator (ICOS) is critical for the development of human T(H)17 cells. Science translational medicine 2010; 2(55):55ra78.
10. Han J M, Patterson S J, Levings M K. The Role of the PI3K Signaling Pathway in CD4(+) T Cell Differentiation and Function. Frontiers in immunology 2012; 3:245.
11. Gigoux M, Shang J, Pak Y, Xu M, Choe J, Mak T W, et al. Inducible costimulator promotes helper T-cell differentiation through phosphoinositide 3-kinase. Proceedings of the National Academy of Sciences of the United States of America 2009; 106(48):20371-6.
12. Vanhaesebroeck B, Guillermet-Guibert J, Graupera M, Bilanges B. The emerging mechanisms of isoform-specific PI3K signalling. Nature reviews Molecular cell biology 2010; 11 (5): 329-41.
13. Fung-Leung W P. Phosphoinositide 3-kinase delta (PI3Kdelta) in leukocyte signaling and function. Cellular signalling 2011; 23(4):603-8.
14. Okkenhaug K, Bilancio A, Farjot G, Priddle H, Sancho S, Peskett E, et al. Impaired B and T cell antigen receptor signaling in p110delta PI 3-kinase mutant mice. Science (New York, N.Y.) 2002; 297(5583):1031-4.
15. Haylock-Jacobs S, Comerford I, Bunting M, Kara E, Townley S, Klingler-Hoffmann M, et al. PI3Kdelta drives the pathogenesis of experimental autoimmune encephalomyelitis by inhibiting effector T cell apoptosis and promoting Th17 differentiation. Journal of autoimmunity 2011; 36(3-4):278-87.
16. Aragoneses-Fenoll L, Montes-Casado M, Ojeda G, Acosta Y Y, Herranz J, Martinez S, et al. ETP-46321, a dual p110alpha/delta class IA phosphoinositide 3-kinase inhibitor modulates T lymphocyte activation and collagen-induced arthritis. Biochemical pharmacology 2016; 106:56-69.
17. Garcon F, Okkenhaug K. PI3Kdelta promotes CD4 T-cell interactions with antigen-presenting cells by increasing LFA-1 binding to ICAM-1. Immunology and cell biology 2016.
18. Chen H, Fu T, Suh W K, Tsavachidou D, Wen S, Gao J, et al. CD4 T cells require ICOS-mediated PI3K signaling to increase T-Bet expression in the setting of anti-CTLA-4 therapy. Cancer immunology research 2014; 2(2):167-76.
19. Simpson T R, Quezada S A, Allison J P. Regulation of CD4 T cell activation and effector function by inducible costimulator (ICOS). Current opinion in immunology 2010; 22(3):326-32.
20. Gattinoni L, Ji Y, Restifo N P. Wnt/beta-catenin signaling in T-cell immunity and cancer immunotherapy. Clinical cancer research: an official journal of the American Association for Cancer Research 2010; 16(19):4695-701.
21. Bauquet A T, Jin H, Paterson A M, Mitsdoerffer M, Ho I C, Sharpe A H, et al. The costimulatory molecule ICOS regulates the expression of c-Maf and IL-21 in the development of follicular T helper cells and TH-17 cells. Nature immunology 2009; 10(2):167-75.
22. van der Windt G J, Everts B, Chang C H, Curtis J D, Freitas T C, Amiel E, et al. Mitochondrial respiratory capacity is a critical regulator of CD8⁺ T cell memory development. Immunity 2012; 36(1):68-78.
23. Mao C D, Byers S W. Cell-context dependent TCF/LEF expression and function: alternative tales of repression, de-repression and activation potentials. Critical reviews in eukaryotic gene expression 2011; 21(3):207-36.
24. Lannutti B J, Meadows S A, Herman S E, Kashishian A, Steiner B, Johnson A J, et al. CAL-101, a p110delta selective phosphatidylinositol-3-kinase inhibitor for the treatment of B-cell malignancies, inhibits PI3K signaling and cellular viability. Blood 2011; 117(2):591-4.
25. Vlahos C J, Matter W F, Hui K Y, Brown R F. A specific inhibitor of phosphatidylinositol 3-kinase, 2-(4-morpholinyl)-8-phenyl-4H-1-benzopyran-4-one (LY294002). The Journal of biological chemistry 1994; 269(7):5241-8.
26. Wang Y, Krivtsov A V, Sinha A U, North T E, Goessling W, Feng Z, et al. The Wnt/beta-catenin pathway is required for the development of leukemia stem cells in AML. Science (New York, N.Y.) 2010; 327(5973):1650-3.
27. Dihlmann S, Klein S, Doeberitz My M. Reduction of beta-catenin/T-cell transcription factor signaling by aspirin and indomethacin is caused by an increased stabilization of phosphorylated beta-catenin. Molecular cancer therapeutics 2003; 2(6):509-16.
28. Lee Y S, Lee K A, Yoon H B, Yoo S A, Park Y W, Chung Y, et al. The Wnt inhibitor secreted Frizzled-Related Protein 1 (sFRP1) promotes human Th17 differentiation. European journal of immunology 2012; 42(10):2564-73.
29. Gattinoni L, Powell D J, Jr., Rosenberg S A, Restifo N P. Adoptive immunotherapy for cancer: building on success. Nature reviews Immunology 2006; 6(5):383-93.
30. Wang J, Yu L, Jiang C, Fu X, Liu X, Wang M, et al. Cerebral ischemia increases bone marrow CD4⁺CD25⁺ FoxP3⁺ regulatory T cells in mice via signals from sympathetic nervous system. Brain, behavior, and immunity 2015; 43:172-83.
31. Ghosh S, Sarkar M, Ghosh T, Guha I, Bhuniya A, Saha A, et al. Neem leaf glycoprotein promotes dual generation of central and effector memory CD8(+) T cells against sarcoma antigen vaccine to induce protective anti-tumor immunity. Molecular immunology 2016; 71:42-53.
32. Crompton J G, Sukumar M, Roychoudhuri R, Clever D, Gros A, Eil R L, et al. Akt inhibition enhances expansion of potent tumor-specific lymphocytes with memory cell characteristics. Cancer research 2015; 75(2):296-305.
33. Abu Eid R, Friedman K M, Mkrtichyan M, Walens A, King W, Janik J, et al. Aktl and −2 inhibition diminishes terminal differentiation and enhances central memory CD8 T-cell proliferation and survival. Oncoimmunology 2015; 4(5):e1005448.
34. van der Waart A B, van de Weem N M, Maas F, Kramer C S, Kester M G, Falkenburg J H, et al. Inhibition of Akt signaling promotes the generation of superior tumor-reactive T cells for adoptive immunotherapy. Blood 2014; 124(23):3490-500.
35. Korn T, Mitsdoerffer M, Croxford A L, Awasthi A, Dardalhon V A, Galileos G, et al. IL-6 controls Th17 immunity in vivo by inhibiting the conversion of conventional T cells into Foxp3+ regulatory T cells. Proceedings of the National Academy of Sciences of the United States of America 2008; 105(47):18460-5.
36. Wu J Q, Seay M, Schulz V P, Hariharan M, Tuck D, Lian J, et al. Tcf7 is an important regulator of the switch of self-renewal and differentiation in a multipotential hematopoietic cell line. PLoS genetics 2012; 8(3):e1002565.
37. Restifo N P. Big bang theory of stem-like T cells confirmed. Blood 2014; 124(4):476-7.
38. Li H, Bradbury J A, Dackor R T, Edin M L, Graves J P, DeGraff L M, et al. Cyclooxygenase-2 regulates Th17 cell differentiation during allergic lung inflammation. American journal of respiratory and critical care medicine 2011; 184(1):37-49.
39. Fan X, Quezada S A, Sepulveda M A, Sharma P, Allison J P. Engagement of the ICOS pathway markedly enhances efficacy of CTLA-4 blockade in cancer immunotherapy. The Journal of experimental medicine 2014; 211(4):715-25.
40. Carthon B C, Wolchok J D, Yuan J, Kamat A, Ng Tang D S, Sun J, et al. Preoperative CTLA-4 blockade: tolerability and immune monitoring in the setting of a presurgical clinical trial. Clinical cancer research: an official journal of the American Association for Cancer Research 2010; 16(10):2861-71.
41. Chen H, Liakou C I, Kamat A, Pettaway C, Ward J F, Tang D N, et al. Anti-CTLA-4 therapy results in higher CD4+ICOShi T cell frequency and IFN-gamma levels in both nonmalignant and malignant prostate tissues. Proceedings of the National Academy of Sciences of the United States of America 2009; 106(8):2729-34.
42. Liakou C I, Kamat A, Tang D N, Chen H, Sun J, Troncoso P, et al. CTLA-4 blockade increases IFNgamma-producing CD4+ICOShi cells to shift the ratio of effector to regulatory T cells in cancer patients. Proceedings of the National Academy of Sciences of the United States of America 2008; 105(39):14987-92.
43. Shen C J, Yang Y X, Han E Q, Cao N, Wang Y F, Wang Y, et al. Chimeric antigen receptor containing ICOS signaling domain mediates specific and efficient antitumor effect of T cells against EGFRvIII expressing glioma. Journal of hematology & oncology 2013; 6:33.
44. Guedan S, Chen X, Madar A, Carpenito C, McGettigan S E, Frigault M J, et al. ICOS-based chimeric antigen receptors program bipolar TH17/TH1 cells. Blood 2014; 124(7): 1070-80.
45. Schmittgen T D, Livak K J. Analyzing real-time PCR data by the comparative C(T) method. Nature protocols 2008; 3(6):1101-8.
U.S. Pat. No. 4,870,287
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,844,905
U.S. Pat. No. 5,846,945
U.S. Pat. No. 5,885,796
U.S. Pat. No. 6,207,156
U.S. Pat. No. 6,225,042
U.S. Pat. No. 6,355,479
U.S. Pat. No. 6,362,001
U.S. Pat. No. 6,410,319
U.S. Pat. No. 6,451,995
U.S. Pat. No. 6,790,662
U.S. Pat. No. 7,070,995
U.S. Pat. No. 7,109,304
U.S. Pat. No. 7,265,209
U.S. Pat. No. 7,354,762
U.S. Pat. No. 7,446,179
U.S. Pat. No. 7,446,190
U.S. Pat. No. 7,446,191
U.S. Pat. No. 8,008,449
U.S. Pat. No. 8,017,114
U.S. Pat. No. 8,119,129
U.S. Pat. No. 8,252,592
U.S. Pat. No. 8,324,353
U.S. Pat. No. 8,329,867
U.S. Pat. No. 8,339,645
U.S. Pat. No. 8,354,509
U.S. Pat. No. 8,398,282
U.S. Pat. No. 8,479,118
U.S. Pat. No. 8,735,553
U.S. Patent Publication No. 2002/131960,
U.S. Patent Publication No. 2005/0260186
U.S. Patent Publication No. 2006/0104968
U.S. Patent Publication No. 2009/0004142
U.S. Patent Publication No. 2009/0017000
U.S. Patent Publication No. 2011/0008369
U.S. Patent Publication No. 2013/0149337
U.S. Patent Publication No. 2013/287748
U.S. Patent Publication No. 2014/022021
U.S. Patent Publication No. 2014/0294898
EP2537416
WO 00/37504
WO 01/14424
WO 98/42752
WO1995/001994
WO1998/042752
WO2000/037504
WO2000/14257
WO2001014424
WO2007/103009
WO2012/129514
WO2013/071154
WO2013/123061
WO2013/126726
WO2013/166321
WO2014/031687
WO2014/055668
WO2014/055668 A1
WO2015/016718
Acosta-Rodriguez et al., Surface phenotype and antigenic specificity of human interleukin 17-producing T helper memory cells. Nat. Immunol. 8, 639-646, 2007.
Austin-Ward and Villaseca, 1998.
Ausubel et al., Current Protocols in Molecular Biology, Greene Publishing Associates and John Wiley & Sons, N Y, 1994.
Barreira da Silva et. al.
Bengsch et al., Human Th17 cells express high levels of enzymatically active dipeptidylpeptidase IV (CD26). J. Immunol. 188, 5438-5447, 2012.
Berger et al., Adoptive transfer of effector CD8⁺ T cells derived from central memory cells establishes persistent T cell memory in primates. J Clin Invest 118: 294-305, 2008.
Betts et al., HIV nonprogressors preferentially maintain highly functional HIV-specific CD8+ T cells. Blood 107, 4781-4789, 2006.
Bowers et al., Th17 cells are refractory to senescence and retain robust antitumor activity after long-term ex vivo expansion. JCI Insight 2, e90772, 2017.
Boyman et al., Selective stimulation of T cell subsets with antibody-cytokine immune complexes. Science 311:1924-1927, 2006.
Bukowski et al., 1998.
Camacho et al., J Clin Oncology 22(145): Abstract No. 2505 (antibody CP-675206), 2004.
Carpenito et al., Control of large, established tumor xenografts with genetically retargeted human T cells containing CD28 and CD137 domains. Proc Natl Acad Sci USA 106:3360-3365, 2009.
Carter et al., 2008.
Chang et al., T helper 17 cells play a critical pathogenic role in lung cancer. Proc. Natl. Acad. Sci. U.S.A. 111, 5664-5669, 2014.
Chothia et al., EMBO J. 7:3745, 1988.
Christodoulides et al., 1998.
Cohen et al., J Immunol. 175:5799-5808, 2005.
Davidson et al., 1998.
Davila et al., PLoS ONE 8(4): e61338, 2013.
Deng et al., Mice lacking p21CIP1/WAF1 undergo normal development, but are defective in G1 checkpoint control, Cell, 82:675-684, 1995.
Fedorov et al., Sci. Transl. Medicine, 5(215), December 2013.
Gattinoni et al., A human memory T cell subset with stem cell-like properties. Nat. Med. 17, 1290-1297, 2011.
Gattinoni et al., Removal of homeostatic cytokine sinks by lymphodepletion enhances the efficacy of adoptively transferred tumor-specific CD8⁺ T cells. J Exp Med 202, 907-912, 2005.(a)
Gattinoni et al., Acquisition of full effector function in vitro paradoxically impairs the in vivo antitumor efficacy of adoptively transferred CD8⁺ T cells. The Journal of clinical investigation 115, 1616-1626 (2005).(b)
Hanibuchi et al., 1998.
Heemskerk et al., Hum Gene Ther. 19:496-510, 2008.
Hellstrand et al., 1998
Hinrichs et al., Human effector CD8⁺ T cells derived from naive rather than memory subsets possess superior traits for adoptive immunotherapy. Blood, 117: 808-814, 2011.
Hollander 2012.
Hui and Hashimoto, 1998.
Hurwitz et al., Proc Natl Acad Sci USA 95(17): 10067-10071, 1998.
Janeway et al, Immunobiology: The Immune System in Health and Disease, 3^{rd} Ed., Current Biology Publications, p. 4:33, 1997
Johnson et al., Blood 114:535-46, 2009.
Johnson et al., Effector C D8⁺ T-cell Engraftment and Antitumor Immunity in Lymphodepleted Hosts Is IL7Ralpha Dependent. Cancer Immunol Res 3, 1364-1374, 2015.
Jores et al., Pwc. Nat'l Acad. Sci. U.S.A. 87:9138, 1990.
Kabat et al., “Sequences of Proteins of Immunological Interest, US Dept. Health and Human Services, Public Health Service National Institutes of Health, 5^{th} ed, 1991.
Kim and Cantor, CD4 T-cell subsets and tumor immunity: the helpful and the not-so-helpful. Cancer Immunol Res 2, 91-98, 2014.
Klebanoff et al., Programming tumor-reactive effector memory CD8⁺ T cells in vitro obviates the requirement for in vivo vaccination. Blood 114, 1776-1783, 2009.
Klebanoff et al., Memory T cell-driven differentiation of naive cells impairs adoptive immunotherapy. J Clin Invest 126: 318-334, 2016.
Laird and Ware, Random-effects models for longitudinal data. Biometrics 38, 963-974, 1982.
Law et al., voom: Precision weights unlock linear model analysis tools for RNA-seq read counts. Genome Biol 15, R29, 2014.
Leal et al., 2014.
Lee et al., Induction and molecular signature of pathogenic TH17 cells. Nat. Immunol. 13, 991-999, 2012.
Lefranc et al., Dev. Comp. Immunol. 27:55, 2003.
Li, Nat Biotechnol. 23:349-354, 2005.
Majchrzak et al., beta-catenin and PI3Kdelta inhibition expands precursor Th17 cells with heightened stemness and antitumor activity, JCI Insight 2, 2017.
Mamonkin et al., Differential roles of KLF4 in the development and differentiation of CD8+ T cells. Immunol Lett 156, 94-101, 2013.
Mellman et al., Nature 480:480-489, 2011.
Mokyr et al. Cancer Res 58:5301-5304, 1998.
Muranski et al., Tumor-specific Th17-polarized cells eradicate large established melanoma. Blood 112, 362-373, 2008.
Pardoll, Nature Rev Cancer 12:252-264, 2012.
Parkhurst et al., Clin Cancer Res. 15: 169-180, 2009.
Paulos et al., The inducible costimulator (ICOS) is critical for the development of human T(H)17 cells. Sci. Transl. Med. 2, 55ra78, 2010.
Paulos et al., Microbial translocation augments the function of adoptively transferred self/tumor-specific CD8+ T cells via TLR4 signaling. J Clin Invest 117, 2197-2204, 2007.
Qin et al., 1998.
Quezada et al., Tumor-reactive CD4(+) T cells develop cytotoxic activity and eradicate large established melanoma after transfer into lymphopenic hosts. The Journal of experimental medicine, 207: 637-650, 2010.
Remington's Pharmaceutical Sciences 22nd edition, 2012
Sadelain et al., Cancer Discov., 3(4): 388-398, April 2013.
Sambrook et al., Molecular Cloning: A Laboratory Manual, 3^{rd} ed., Cold Spring Harbor Press, Cold Spring Harbor, N.Y. 2001.
Sanna et al., ILPIP, a novel anti-apoptotic protein that enhances XIAP-mediated activation of JNK1 and protection against apoptosis. J Biol Chem 277, 30454-30462, 2002.
Schluns et al., Interleukin-7 mediates the homeostasis of naive and memory CD8 T cells in vivo. Nat Immunol 1:426-432, 2000.
Shah et al., 2013
Singh et al., 2008.
Singh et al., 2011.
Sommermeyer et al., Chimeric antigen receptor-modified T cells derived from defined CD8⁺ and CD4+ subsets confer superior antitumor reactivity in vivo. Leukemia 30: 492-500, 2016.
Teicher 2009.
Teicher 2014.
Terakuraet et al., Blood. 1:72-82, 2012.
Turtle et al., Curr. Opin. Immunol., 24(5): 633-39, October 2012.
Varela-Rohena et al., Nat Med. 14: 1390-1395, 2008.
Wang et al., J Immunother. 35(9):689-701, 2012.
Wang et al., Phase 1 studies of central memory-derived CD19 CAR T-cell therapy following autologous HSCT in patients with B-cell NHL. Blood 127: 2980-2990, 2016.
Wu et al., Cancer, 18(2): 160-75, March, 2012.
Xie et al., Naive tumor-specific CD4(+) T cells differentiated in vivo eradicate established melanoma, The Journal of experimental medicine, 207: 651-667, 2010.

What is claimed is:

1. An in vitro method for producing enhanced immune cells comprising:
(a) obtaining a starting population of immune cells; and
(b) culturing the cells in the presence of an inhibitor of PI3K/Akt signaling and/or an inhibitor of Wnt/β-catenin signaling, thereby obtaining enhanced immune cells.

2. The method of claim 1, wherein the immune cells comprise T cells, NK cells or macrophages.

3. The method of claim 2, wherein the immune calls comprise CD4⁺ T cells or CD8 T cells

4. The method of claim 3, wherein the immune cells comprise Th17 cells.

5. The method of claim 1, wherein the immune cells are cultured in the presence of an inhibitor of PI3K/Akt signaling and an inhibitor of Wnt/β-catenin signaling.

6. An engineered immune cell comprising a genetic disruption of a gene in the PI3K/Akt pathway and/or the Wnt/β-catenin pathway.

7. The engineered cell of claim 6, comprising a genetic disruption of a gene in the PI3K/Akt pathway and the Wnt/β-catenin pathway.

8. The engineered cell of claim 6, wherein the cell is a T cell, NK cell or macrophage.

9. The engineered cell of claim 8, wherein the cell is a CD4⁺ T cell or CD8 T cell.

10. The engineered cell of claim 6, wherein the disruption was produced with a zinc finger nuclease, a transposase or a CRISPR construct.

11. An in vitro method for producing enhanced inducible costimulator (ICOS)-stimulated Th17 cells comprising:
(a) obtaining a starting population of ICOS-stimulated Th17 cells; and
(b) culturing the Th17 cells in the presence of an inhibitor of PI3K/Akt signaling and/or an inhibitor of Wnt/β-catenin signaling, thereby obtaining enhanced ICOS-stimulated Th17 cells.

12. The method of claim 1 or 11, wherein the inhibitor of PI3K/Akt signaling is an inhibitor of p110δ.

13. The method of claim 1 or 11, wherein the inhibitor of PI3K/Akt signaling is a siRNA short hairpin RNA.

14. The method of claim 1 or 11, wherein the inhibitor of PI3K/Akt signaling disrupts a gene in the PI3K/Akt pathway.

15. The method of claim 14, wherein the inhibitor is a zinc finger nuclease, a transposase or a CRISPR construct.

16. The method of claim 12, wherein the inhibitor of p110δ is CAL-101.

17. The method of claim 1 or 11, wherein the inhibitor of Wnt/β-catenin signaling is an inhibitor of β-catenin.

18. The method of claim 1 or 11, wherein the inhibitor of Wnt/β-catenin signaling is a siRNA short hairpin RNA.

19. The method of claim 1 or 11, wherein the inhibitor of Wnt/β-catenin signaling disrupts a gene in the PI3K/Akt pathway.

20. The method of claim 19, wherein the inhibitor is a zinc finger nuclease, a transposase or a CRISPR construct.

21. The method of claim 17, wherein the inhibitor of β-catenin is indomethacin.

22. The method of claim 21, wherein the indomethacin is present at a concentration of 50 to 100 μM.

23. The method of claim 16, wherein the CAL-101 is present at a concentration of 5 to 15 μM.

24. The method of claim 1 or 11, wherein culturing of step (b) is for 4 to 10 days.

25. The method of claim 1 or 11, wherein the enhanced cells exhibit an increased ability for engraftment, persistence, and/or antitumor activity in vivo.

26. The method of claim 1 or 11, wherein the enhanced cells have decreased expression of RORγt, cMaf and/or STAT-3.

27. The method of claim 1, wherein the enhanced ICOS-stimulated Th17 cells have an increased percentage of CD44^{high},CD62L^{high} cells as compared to the starting population of ICOS-stimulated Th17 cells.

28. The method of claim 1, wherein obtaining a starting population of ICOS-stimulated Th17 cells comprises programming T cells to a Th17 phenotype and stimulating the Th17 cells with ICOS.

29. The method of claim 28, wherein programming comprises culturing the cells in the presence of IL-1β, IL-6, IL-21, TGFβ, IL-4, IFNγ, IL-2, and/or IL-23.

30. The method of claim 28, wherein the T cells are CD4⁺ and/or CD8⁺ T cells.

31. The method of claim 28, wherein stimulating with ICOS comprises culturing the population of Th17 cells in a culture comprising anti-ICOS coated beads.

32. The method of claim 1 or 11, further comprising stimulating with one or more co-stimulatory agents selected from the group consisting of 41BB, CD28, CD40L, OX40, a PD-1 inhibitor, and a CTLA4 inhibitor.

33. The method of claim 31, wherein the beads are magnetic beads.

34. The method of claim 1 or 11, wherein the culture further comprises anti-CD3 beads.

35. The method of claim 1 or 11, wherein the culture further comprises at least one growth factor.

36. The method of claim 1 or 11, wherein the at least one growth factor is IL-2.

37. The method of claim 1 or 11, wherein the culturing is for 5 day to 10 days.

38. The method of claim 1 or 11, wherein the cells are isolated from peripheral blood, cord blood, or the spleen.

39. The method of claim 1 or 11, wherein the cells are isolated from peripheral blood mononuclear cells.

40. The method of claim 1 or 11, wherein the cells are engineered to express a T cell receptor (TCR) or chimeric antigen receptor (CAR) receptor.

41. The method of claim 40, wherein the TCR or CAR comprises an intracellular signaling domain, a transmembrane domain, and/or an extracellular domain comprising an antigen binding region.

42. The method of claim 41, wherein the antigen binding region is an F(ab′)2, Fab′, Fab, Fv, or scFv.

43. The method of claim 41, wherein the intracellular signaling domain is a T-lymphocyte activation domain.

44. The method of claim 41, wherein the intracellular signaling domain comprises CD3, CD28, OX40/CD134, 4-1BB/CD137, FccRIγ, ICOS/CD278, ILRB/CD122, IL-2RG/CD132, DAP molecules, CD70, cytokine receptor, CD40, or a combination thereof.

45. The method of claim 25, wherein the intracellular signaling domain comprises CD3 and 4-1BB/CD137.

46. The method of claim 41, wherein the transmembrane domain comprises CD28 transmembrane domain, IgG4Fc hinge, Fc regions, CD4 transmembrane domain, the CD3ζ transmembrane domain, cysteine mutated human CD3ζ domain, CD16 transmembrane domain, CD8 transmembrane domain, or erythropoietin receptor transmembrane domain.

47. The method of claim 41, wherein the antigen binding region binds a tumor associated antigen.

48. The method of claim 47, wherein the tumor associated antigen is selected from the group consisting of tEGFR, Her2, CD19, CD20, CD22, mesothelin, CEA, CD23, CD24, CD30, CD33, CD38, CD44, EGFR, EGP-2, EGP-4, EPHa2, ErbB2, FBP, MAGE-A1, MUC1, NY-ESO-1, and MART-1.

49. The method of claim 1, wherein the enhanced ICOS-stimulated Th17 cells have decreased expression of FoxP3 and/or CD25.

50. The method of claim 1, wherein the enhanced ICOS-stimulated Th17 cells have a central memory phenotype.

51. The method of claim 1, wherein the enhanced ICOS-stimulated Th17 cells are capable of long-term engraftment in a mammal.

52. The method of claim 51, wherein the mammal is a human.

53. An isolated cell population comprising enhanced immune cells produced according to the methods of any one of claims 1-52.

54. A method of treating cancer in a subject comprising administering an effective amount of the enhanced immune cells of claim 53 to the subject.

55. The method of claim 54, wherein the cancer is melanoma.

56. The method of claim 54, further comprising performing total body irradiation to the subject prior to administering the enhanced ICOS-stimulated Th17 cells.

57. The method of claim 54, wherein the enhanced Th17 cells exhibit increased tumor regression as compared to the starting population of Th17 cells.

58. The method of claim 45, wherein the Th17 cells are autologous.

59. The method of claim 54, further comprising lymphodepletion of the subject prior to administration of the Th17 cells.

60. The method of claim 59, wherein lymphodepletion comprises administration of cyclophosphamide and/or fludarabine.

61. The method of claim 54, further comprising administering at least a second therapeutic agent.

62. The method of claim 61, wherein the at least a second therapeutic agent comprises CD8⁺ T cells.

63. The method of claim 61, wherein the at least a second therapeutic agent comprises chemotherapy, immunotherapy, surgery, radiotherapy, or biotherapy.

64. The method of claim 63, wherein the immunotherapy is an immune checkpoint inhibitor.

65. The method of claim 61, wherein the Th17 cells and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion.

66. The method of claim 54, wherein the cancer is bladder cancer, breast cancer, clear cell kidney cancer, head/neck squamous cell carcinoma, lung squamous cell carcinoma, melanoma, non-small-cell lung cancer (NSCLC), ovarian cancer, pancreatic cancer, prostate cancer, renal cell cancer, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL).

67. The method of claim 54, wherein said subject is a human subject.
